(19) United States
(12) Patent Application Publication
(10) Pub. No.: US 2014/0328799 A1
(43) Pub. Date: Nov. 6, 2014

(54) COMPOSITIONS USEFUL FOR THE TREATMENT OF VIRAL DISEASES

(71) Applicants: Steven S. Carroll, Yardley, PA (US); John A. McCauley, Maple Glen, PA (US); David B. Olsen, Lansdale, PA (US); Vincenzo Summa, Pomezia (IT); Michael Rowley, Luzern (CH); Mark W. Stahlhut, North Wales, PA (US); Daria Hazuda, West Point, PA (US)

(72) Inventors: Steven S. Carroll, Yardley, PA (US); John A. McCauley, Maple Glen, PA (US); David B. Olsen, Lansdale, PA (US); Vincenzo Summa, Pomezia (IT); Michael Rowley, Luzern (CH); Mark W. Stahlhut, North Wales, PA (US); Daria Hazuda, West Point, PA (US)

(21) Appl. No.: 14/355,363

(22) PCT Filed: Oct. 26, 2012
(86) PCT No.: PCT/US12/62145

§ 371 Date: Apr. 30, 2014

Related U.S. Application Data
(60) Provisional application No. 61/553,677, filed on Oct. 31, 2011.

(51) Int. Cl. A61K 38/06; A61K 31/553; A61K 31/519; A61K 31/4178; A61K 31/7064

(52) U.S. Cl. CPC A61K 38/06; A61K 31/4178; A61K 31/7064; A61K 31/519; A61K 31/553
USPC 424/85.7; 514/4.3

(57) ABSTRACT

The present invention is directed to compositions comprising inhibitors of hepatitis C virus (HCV) protease and one or more additional therapeutically effective agents. Uses of such compositions as HCV inhibitors and methods of treating infection by HCV by administration of such compositions are also disclosed.

### FIELD OF THE INVENTION

The present invention is directed to compositions comprising inhibitors of hepatitis C virus (HCV) protease and one or more additional therapeutically effective agents. Uses of such compositions as HCV inhibitors and methods of treating infection by HCV by administration of such compositions are also disclosed.

### BACKGROUND OF THE INVENTION

Hepatitis C virus (HCV) infection is a major health problem that leads to chronic liver disease, such as cirrhosis and hepatocellular carcinoma, in a substantial number of infected individuals. The HCV virion is an enveloped positive-strand RNA virus with a single oligoribonucleotide genomic sequence of about 9600 bases which encodes a polyprotein of about 3,010 amino acids. The protein products of the HCV gene consist of the structural proteins C, E1, and E2, and the non-structural proteins NS2, NS3, NS4A and NS4B, and NS5A and NS5B. The nonstructural (NS) proteins are believed to provide the catalytic machinery for viral replication. The NS3 protease releases NS5B, the RNA-dependent RNA polymerase from the polyprotein chain. HCV NS5B polymerase is required for the synthesis of a double-stranded RNA from a single-stranded viral RNA that serves as a template in the replication cycle of HCV. NS3 protease and NS5B polymerase are therefore considered to be essential components in the HCV replication complex. See K. Ishii, et al., “Expression of Hepatitis C Virus NS5B Protein: Characterization of Its RNA Polymerase Activity and RNA Binding,” Hepatology, 29: 1227-1235 (1999); V. Lohmann, et al., “Biochemical and Kinetic Analyses of NS5B RNA-Dependent RNA Polymerase of the Hepatitis C Virus,” Virology, 249: 108-118 (1998).

Thus, the NS3 serine protease (amino acid residues 1-180) and the NS5B RNA-dependent RNA polymerase have been identified as targets for therapeutic intervention, along with the NS2-3 metalloprotease, the NS3 helicase (full length), the NS4A protease cofactor, the NS4B membrane protein, and the NS5A zinc metalloprotein. Inhibition of HCV NS3 protease and/or HCV NS5B polymerase prevents formation of the double-stranded HCV RNA and therefore constitutes an attractive approach to the development of HCV-specific antiviral therapies.

The use of combination therapy can provide an increased level of inhibition of viral replication that can increase the likelihood of achieving sustained viral response in a patient receiving treatment. The use of a combination of two or more compounds can generate a greater degree of inhibition of viral replication than the use of the same dose of each compound when used individually. Another advantage to the use of combination therapy relates to the development of viral resistance to inhibition by a compound, characterized by a reduced susceptibility to inhibition. Viral variants with sequence mutations can arise during dosing of the compound to an infected individual. These viral variants can have reduced susceptibility to inhibition by a particular compound, and yet remain susceptible to inhibition by another compound or class of compounds that act through a different mechanism, or act through inhibition of another viral enzyme. The use of combination therapy can reduce or eliminate the development of resistance and improve the likelihood of achieving sustained viral response.

There is a clear need to develop effective combination therapeutics for treatment of HCV infection. Specifically, there is a need to develop combinations that are useful for treating HCV-infected patients and compounds that inhibit HCV viral replication.

### SUMMARY OF THE INVENTION

The present invention provides pharmaceutical compositions (the “HCV Inhibitory Compositions”) comprising: (i) a pharmaceutically acceptable carrier; (ii) a compound selected from Table 1:

TABLE 1

| | |
|---|---|
| 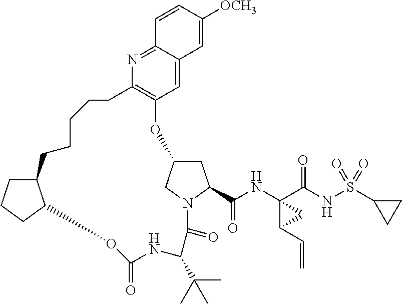 | 1 |
| 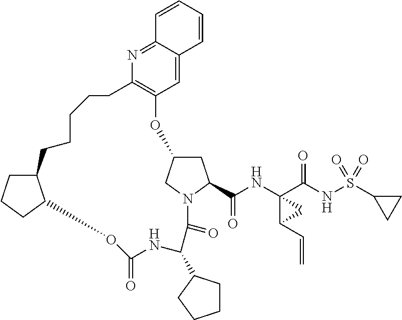 | 2 |
| 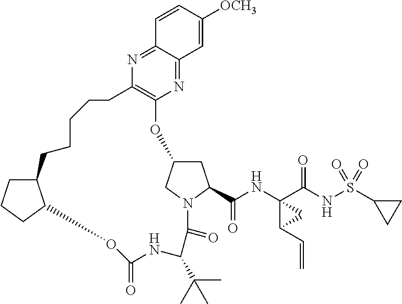 | 3 |
| 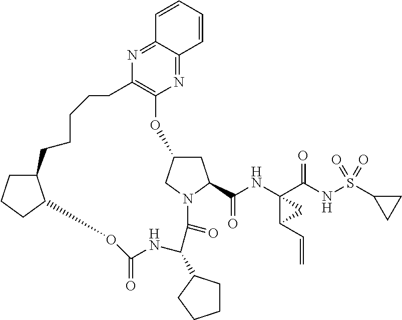 | 4 |
| 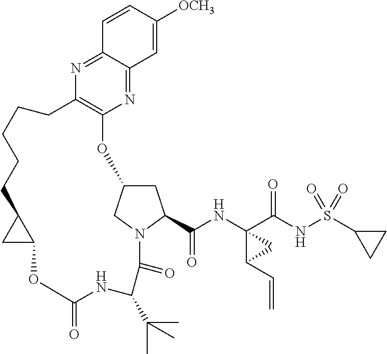 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)NS(=O)(=O)C1CC1] | 5 |
| 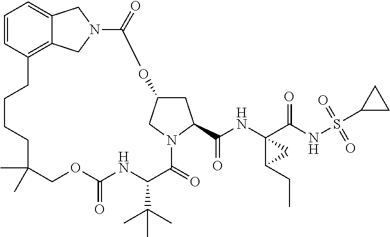 [SMILES: CC[C@@H]1C[C@]1(NC(=O)[C@@H]1C[C@@H]2CN1C(=O)[C@H](C(C)(C)C)NC(=O)OCC(C)(C)CCCCc1cccc3c1CN(C3)C(=O)O2)C(=O)NS(=O)(=O)C1CC1] | 6 |
| 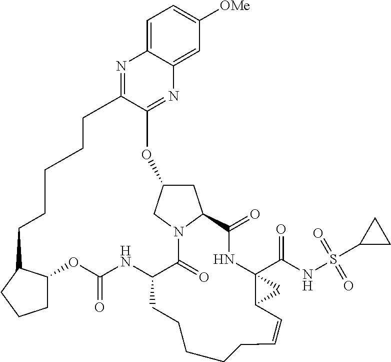 | 7 |
| 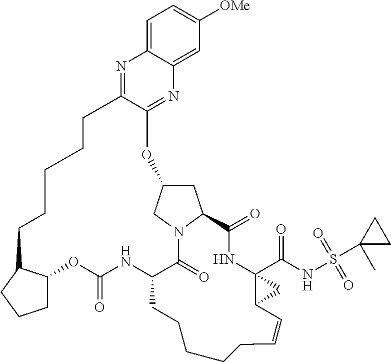 | 8 |
| 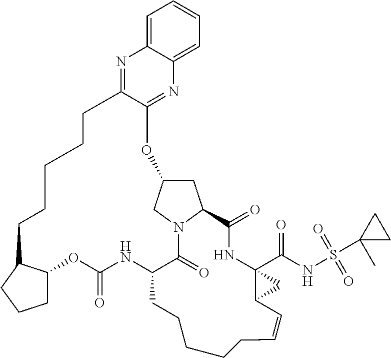 | 9 |
| 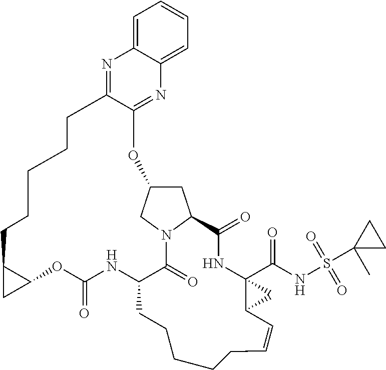 [SMILES: CC1(S(=O)(=O)NC(=O)[C@@]23C[C@H]2/C=C\CCCCC[C@@H]2NC(=O)O[C@@H]4C[C@H]4CCCCCc4nc5ccccc5nc4O[C@@H]4C[C@@H](C(=O)N3)N(C4)C2=O)CC1] | 10 |
| 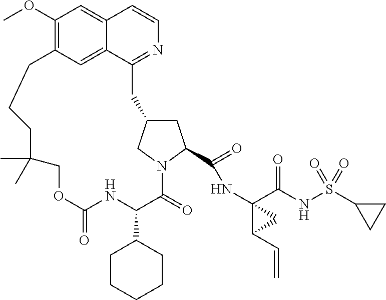 [SMILES: C=C[C@@H]1C[C@]1(NC(=O)[C@@H]1C[C@H]2Cc3nccc4cc(OC)c(cc34)CCCC(C)(C)COC(=O)N[C@@H](C3CCCCC3)C(=O)N1C2)C(=O)NS(=O)(=O)C1CC1] | 11 |
| 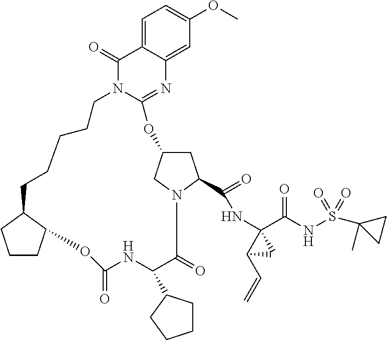 [SMILES: C=C[C@@H]1C[C@]1(NC(=O)[C@@H]1C[C@@H]2CN1C(=O)[C@H](C1CCCC1)NC(=O)O[C@@H]1CCC[C@H]1CCCCCn1c(nc3cc(OC)ccc3c1=O)O2)C(=O)NS(=O)(=O)C1(C)CC1] | 12 |
| 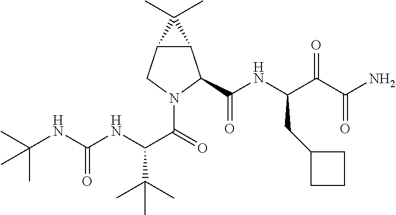 [SMILES: CC(C)(C)NC(=O)N[C@H](C(=O)N1C[C@H]2[C@@H]([C@H]1C(=O)N[C@H](CC1CCC1)C(=O)C(N)=O)C2(C)C)C(C)(C)C] | 13 |
| 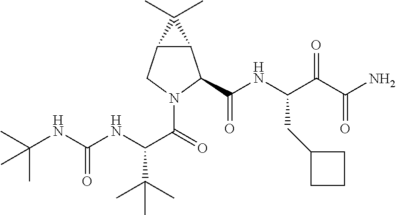 [SMILES: CC(C)(C)NC(=O)N[C@H](C(=O)N1C[C@H]2[C@@H]([C@H]1C(=O)N[C@@H](CC1CCC1)C(=O)C(N)=O)C2(C)C)C(C)(C)C] | 14 |

or a pharmaceutically acceptable salt thereof; and (iii) one or more Primary Additional Therapeutic Agents, or a pharmaceutically acceptable salt thereof, wherein the one or more Primary Additional Therapeutic Agents are selected from the group consisting of HCV protease inhibitors, HCV polymerase inhibitors, HCV NS4A inhibitors and HCV NS5A inhibitors, wherein the one or more Primary Additional Therapeutic Agents do not comprise a Compound of Table 1, and wherein the amounts of the Compound of Table 1 and the one or more Primary Additional Therapeutic Agents are together effective to treat HCV infection in a patient, such that when the one or more Primary Additional Therapeutic Agents comprise an HCV NS5A inhibitor, the HCV NS5A inhibitor is not one of the following compounds:

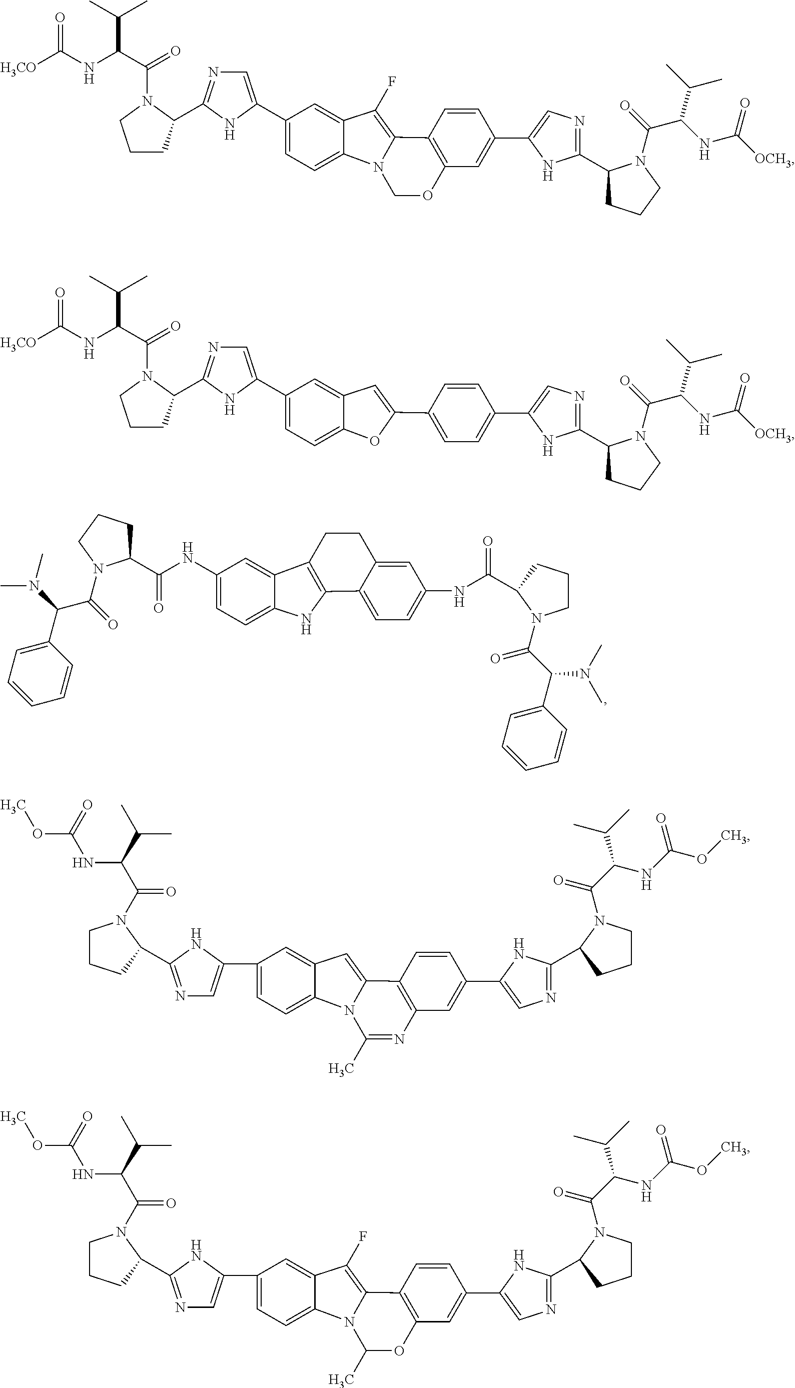

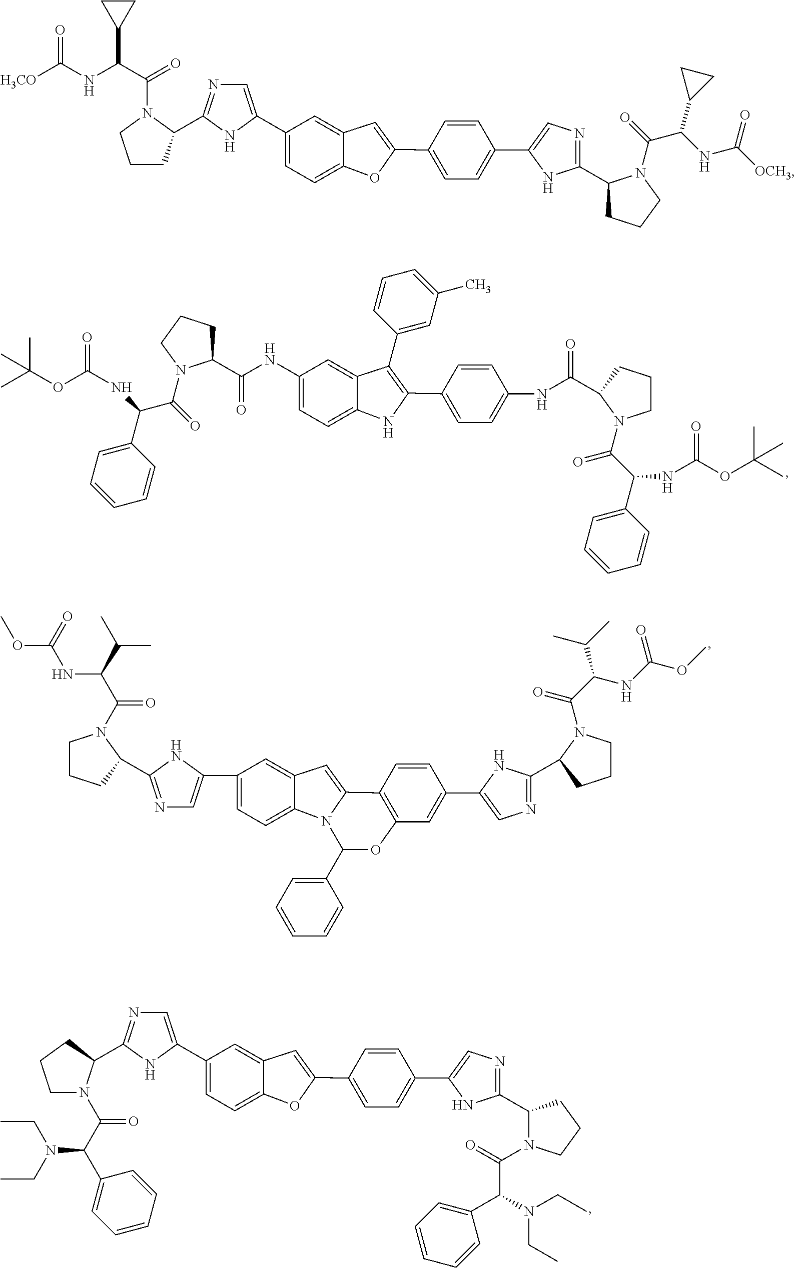

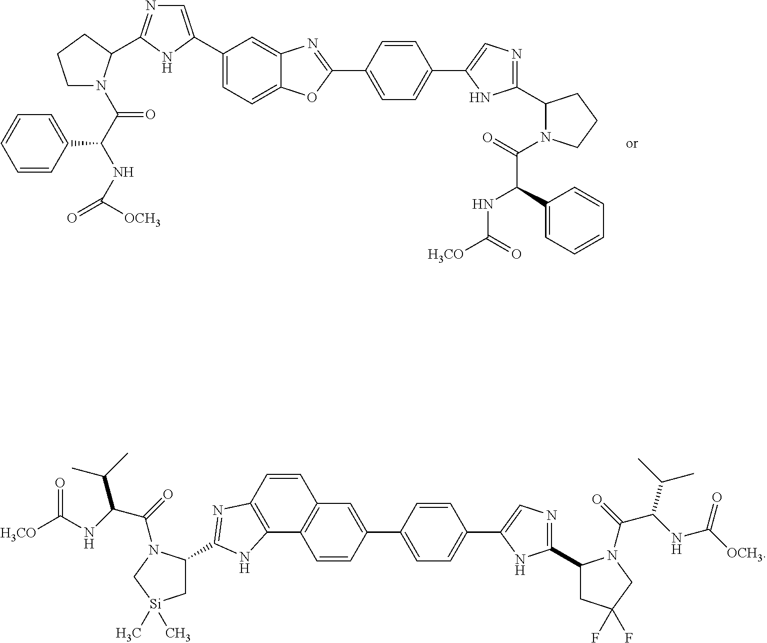

The HCV Inhibitory Compositions can optionally further comprise one or more Secondary Additional Therapeutic Agents, as defined below herein.

The HCV Inhibitory Compositions can be useful, for example, for inhibiting HCV viral replication or replicon activity, and for treating or preventing HCV infection in a patient. Without being bound by any specific theory, it is believed that the Compounds of Table 1 inhibit HCV viral replication by inhibiting HCV RNA Protease.

The details of the invention are set forth in the accompanying detailed description below.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### BRIEF DESCRIPTION OF THE DRAWINGS

FIG. 1 shows the results of MACSYNERGY™ II analysis of inhibition in an HCV replicon assay by a combination of Compound 6 and Compound T1. The x-axis represents concentration of Compound 6 (μM); the y-axis represents concentration of Compound T1 (μM); and the z-axis (vertical axis) represents the degree of synergy exhibited by the combination versus what would be expected from an additive effect. The graph indicates the presence of synergy for the combinations of Compound 6 and Compound T1 at concentrations indicated by the peak.

FIG. 2 shows the results of analysis of the inhibition in an HCV replicon assay by a combination of Compound 6 and Compound T1, wherein the analysis was carried out using the method described in Barton et al., Biometrics 1993 49, 95-105. The left side margin represents concentration of Compound 6 (μM); the right side margin represents the percent inhibition of Compound 6 at the indicated concentration relative to control (i.e., no inhibitor present); the top margin represents the concentration of Compound T1 (μM); and the bottom margin represents the percent inhibition of Compound T1 at the indicated concentration relative to control (i.e., no inhibitor present). The data set forth in the table can be interpreted as follows: “Sat” means 100% saturation and no interpretation possible; “Add” means that an additive effect and a potential synergistic effect is present; and “Inert” means that a measurable, but not significant, deviation from additivity was noted. The numbers next to these designations indicate the percent inhibition measured for the combination relative to control (i.e., no inhibitor present).

FIG. 3 shows the results of MACSYNERGY™ II analysis of inhibition in an HCV replicon assay by a combination of Compound 6 and Compound T2. The x-axis represents concentration of Compound T2 (μM); the y-axis represents concentration of Compound 6 (μM); and the z-axis (vertical axis) represents the degree of synergy exhibited by the combination versus what would be expected from an additive effect. The graph indicates the presence of synergy for the combinations of Compound 6 and Compound T2 at concentrations indicated by the peak(s).

FIG. 4 shows the results of analysis of the inhibition in an HCV replicon assay by a combination of Compound 6 and Compound T2, wherein the analysis was carried out using the method described in Barton et al., Biometrics 1993 49, 95-105. The left side margin represents concentration of Compound 6 (μM); the right side margin represents the percent inhibition of Compound 6 at the indicated concentration relative to control (i.e., no inhibitor present); the top margin represents the concentration of Compound T2 (μM); and the bottom margin represents the percent inhibition of Compound T2 at the indicated concentration relative to control (i.e., no inhibitor present). The data set forth in the table can be interpreted as follows: “Sat” means 100% saturation and no interpretation possible; “Add” means that an additive effect and a potential synergistic effect is present; and “Syn” means that a synergistic effect is present. The numbers next to these designations indicate the percent inhibition measured for the combination relative to control (i.e., no inhibitor present).

FIG. 5 shows the results of MACSYNERGY™ II analysis of inhibition in an HCV replicon assay by a combination of Compound 6 and Compound T3. The x-axis represents concentration of Compound T3 (μM); the y-axis represents concentration of Compound 6 (μM); and the z-axis (vertical axis) represents the degree of synergy exhibited by the combination versus what would be expected from an additive effect.

FIG. 6 shows the results of analysis of the inhibition in an HCV replicon assay by a combination of Compound 6 and Compound T3, wherein the analysis was carried out using the method described in Barton et al., Biometrics 1993 49, 95-105. The left side margin represents concentration of Compound 6 (μM); the right side margin represents the percent inhibition of Compound 6 at the indicated concentration relative to control (i.e., no inhibitor present); the top margin represents the concentration of Compound T3 (μM); and the bottom margin represents the percent inhibition of Compound T3 at the indicated concentration relative to control (i.e., no inhibitor present). The term “Add” in the table means that an additive effect and a potential synergistic effect is present and the numbers next to this term indicate the percent inhibition measured for the combination relative to control (i.e., no inhibitor present). The graph indicates synergy present for combinations of Compound 6 and Compound T3 at concentrations indicated by the peak(s).

### DETAILED DESCRIPTION OF THE INVENTION

The present invention relates to HCV Inhibitory Compositions and methods of using the HCV Inhibitory Compositions for treating or preventing a viral infection in a patient.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated.

As used herein, and throughout this disclosure, the following terms, unless otherwise indicated, shall be understood to have the following meanings:

A “patient” is a human or non-human mammal. In one embodiment, a patient is a human. In another embodiment, a patient is a chimpanzee.

The term “effective amount” as used herein, refers to an amount of HCV Inhibitory Composition and one or more additional therapeutic agents, or a composition thereof that is effective in producing the desired therapeutic, ameliorative, inhibitory or preventative effect when administered to a patient suffering from a viral infection or virus-related disorder. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to an HCV viral infection or HCV-virus related disorder, refers to reducing the likelihood of HCV infection.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a HCV Inhibitory Composition or a pharmaceutically acceptable salt or solvate of the compound. The transformation may occur by various mechanisms (e.g., by metabolic or chemical processes), such as, for example, through hydrolysis in blood.

If a HCV Inhibitory Composition incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N- or di-N,N—(C₁-C₆)alkylaminoalkyl; —C(Y⁴)Y⁵ wherein Y⁴ is H or methyl and Y⁵ is mono-N- or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁ alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al., J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al., AAPS PharmSciTechours., 5(1), article 12 (2004); and A. L. Bingham et al., Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The HCV Inhibitory Compositions can form salts which are also within the scope of this invention. Reference to a HCV Inhibitory Composition herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a HCV Inhibitory Composition contains both a basic moiety, such as, but not limited to a pyridine or imiclazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Table 1 may be formed, for example, by reacting a HCV Inhibitory Composition with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, dihydrochlorides, hydrobromides, hydroiodides, lactates, maleates, methanesulfonates (“mesylates”), dimesylates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference thereto.

In one embodiment, the HCV Inhibitory Compositions are in the form of a dihydrochloride salt. In another embodiment, the HCV Inhibitory Compositions are in the form of a dimesylate salt.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quartemized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the HCV Inhibitory Compositions may be atropisomers (e.g., substituted biaryls) and are considered as part of this invention. Enantiomers can also be directly separated using chiral chromatographic techniques.

It is also possible that the HCV Inhibitory Compositions may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a HCV Inhibitory Composition incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Table 1, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the Compounds of Table 1. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Table 1 can be prepared without undue experimentation by conventional techniques well known to those skilled in the art or by processes analogous to those described in the Schemes and Examples herein using appropriate isotopically-enriched reagents and/or intermediates. In one embodiment, a Compound of Table 1 has one or more of its hydrogen atoms replaced with deuterium.

Polymorphic forms of the components of the HCV Inhibitory Compositions, and of the salts, solvates, hydrates, esters and prodrugs of the components of the HCV Inhibitory Compositions are intended to be included in the present invention.

### The HCV Inhibitory Compositions

The present invention provides HCV Inhibitory Compositions comprising: (i) a pharmaceutically acceptable carrier; (ii) a compound selected from Table 1 or a pharmaceutically acceptable salt thereof; and (iii) one or more Primary Additional Therapeutic Agents, or a pharmaceutically acceptable salt thereof, wherein the amounts of the Compound of Table 1 and the one or more Primary Additional Therapeutic Agents are together effective to treat a viral infection in a patient.

In one embodiment, the present invention provides HCV Inhibitory Compositions comprising: (i) a pharmaceutically acceptable carrier; (ii) a compound selected from Table 1 or a pharmaceutically acceptable salt thereof; (iii) one or more Primary Additional Therapeutic Agents or a pharmaceutically acceptable salt thereof; and (iv) one or more Secondary Additional Therapeutic Agents or a pharmaceutically acceptable salt thereof wherein the amounts of the Compound of Table 1, the one or more Primary Additional Therapeutic Agents and the one or more Secondary Additional Therapeutic Agents are together effective to treat a viral infection in a patient.

In one embodiment, one or more of the components of the HCV Inhibitory Compositions are in substantially purified form. In another embodiment, all of the components of the HCV Inhibitory Compositions are in substantially purified form.

In another embodiment, the present invention provides a HCV Inhibitory Composition of the present invention for use in (i) inhibiting HCV replication or (ii) treating HCV infection and/or reducing the likelihood or severity of symptoms of HCV infection. In these uses, the HCV Inhibitory Compositions of the present invention can optionally be employed in combination with one or more Secondary Additional Therapeutic Agents, which are defined below herein.

In another embodiment, the present invention also includes a HCV Inhibitory Composition of the present invention for use (i) in, (ii) as a medicament for, or (iii) in the preparation of a medicament for: (a) medicine, (b) inhibiting HCV replication or (c) treating HCV infection and/or reducing the likelihood or severity of symptoms of HCV infection. In these uses, the HCV Inhibitory Compositions of the present invention can optionally be employed in combination with one or more Secondary Additional Therapeutic Agents, which are defined below herein.

### Uses of the HCV Inhibitory Combinations

The HCV Inhibitory Compositions are useful in human and veterinary medicine for treating or preventing a viral infection in a patient. In one embodiment, the HCV Inhibitory Compositions can be inhibitors of viral replication. In another embodiment, the HCV Inhibitory Compositions can be inhibitors of HCV replication. Accordingly, the HCV Inhibitory Compositions are useful for treating viral infections, such as HCV. In accordance with the invention, the HCV Inhibitory Compositions can be administered to a patient in need of treatment or prevention of a viral infection.

Accordingly, in one embodiment, the invention provides methods for treating a viral infection in a patient comprising administering to the patient an effective amount of an HCV Inhibitory Composition. When used to treat a viral infection, the components of the HCV Inhibitory Compositions of the present invention can be administered together in a single dosage form, or the components can be administered separately and optionally, at different times.

### Treatment of Prevention of a Flaviviridae Virus

The HCV Inhibitory Compositions can be useful in combination with one or more additional therapeutic agents for treating or preventing a viral infection caused by the Flaviviridae family of viruses.

Examples of Flaviviridae infections that can be treated or prevented using the present methods include but are not limited to, dengue fever, Japanese encephalitis, Kyasanur Forest disease, Murray Valley encephalitis, St. Louis encephalitis, Tick-borne encephalitis, West Nile encephalitis, yellow fever and Hepatitis C Virus (HCV) infection.

In one embodiment, the Flaviviridae infection being treated is hepatitis C virus infection.

### Treatment or Prevention of HCV Infection

The HCV Inhibitory Compositions can be useful for the inhibition of HCV (e.g., HCV NS5A), the treatment of HCV infection and/or reduction of the likelihood or severity of symptoms of HCV infection and the inhibition of HCV viral replication and/or HCV viral production in a cell-based system. For example, the HCV Inhibitory Compositions are useful in treating infection by HCV after suspected past exposure to HCV by such means as blood transfusion, exchange of body fluids, bites, accidental needle stick, or exposure to patient blood during surgery or other medical procedures.

In one embodiment, the hepatitis C infection is acute hepatitis C. In another embodiment, the hepatitis C infection is chronic hepatitis C.

Accordingly, in one embodiment, the present invention provides methods for treating HCV infection in a patient, the methods comprising administering to the patient an effective amount of a HCV Inhibitory Composition. In a specific embodiment, the amounts administered of the components of the HCV Inhibitory Composition are together effective to treat or prevent infection by HCV in the patient. In another specific embodiment, the amounts administered of the components of the HCV Inhibitory Composition are together effective to inhibit HCV viral replication and/or viral production in the patient. In another embodiment, the amounts administered of the components of the Inhibitory Composition are those that render each of the components alone effective.

The compositions and combinations of the present invention can be useful for treating a patient suffering from infection related to any HCV genotype. HCV types and subtypes may differ in their antigenicity, level of viremia, severity of disease produced, and response to interferon therapy as described in Holland et al., Pathology, 30(2):192-195 (1998). The nomenclature set forth in Simmonds et al., J Gen Virol, 74(Pt11):2391-2399 (1993) is widely used and classifies isolates into six major genotypes, 1 through 6, with two or more related subtypes, e.g., 1a and 1b. Additional genotypes 7-10 and 11 have been proposed, however the phylogenetic basis on which this classification is based has been questioned, and thus types 7, 8, 9 and 11 isolates have been reassigned as type 6, and type 10 isolates as type 3 (see Lamballerie et al., J Gen Virol, 78(Pt1):45-51 (1997)). The major genotypes have been defined as having sequence similarities of between 55 and 72% (mean 64.5%), and subtypes within types as having 75%-86% similarity (mean 80%) when sequenced in the NS-5 region (see Simmonds et al., J Gen Virol, 75(Pt 5):1053-1061 (1994)).

### The Additional Therapeutic Agents

The present invention provides HCV Inhibitory Compositions and methods of use thereof for treating or preventing a viral disease in a patient. The HCV Inhibitory Compositions comprise a pharmaceutically acceptable carrier, a Compound of Table 1 and one or more Primary Additional Therapeutic Agents

In one embodiment, the present invention provides methods for treating a viral infection in a patient, the method comprising administering to the patient: (i) a Compound of Table 1 or a pharmaceutically acceptable salt thereof, and (ii) one or more Primary Additional Therapeutic Agents or a pharmaceutically acceptable salt thereof, wherein the amounts administered are together effective to treat or prevent a viral infection.

In another embodiment, the present invention provides methods for treating a viral infection in a patient, the method comprising administering to the patient: (i) a Compound of Table 1 or a pharmaceutically acceptable salt thereof; (ii) one or more Primary Additional Therapeutic Agents or a pharmaceutically acceptable salt thereof; and (iii) one or more Secondary Additional Therapeutic Agents or a pharmaceutically acceptable salt thereof, wherein the amounts administered are together effective to treat or prevent a viral infection.

When administering a combination therapy of the present invention to a patient, the active agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a HCV Inhibitory Composition and a Primary Additional Therapeutic Agent may be present in fixed amounts (dosage amounts) in a single dosage unit (e.g., a capsule, a tablet and the like).

In one embodiment, the Compound of Table 1 is administered during a time when the Primary Additional Therapeutic Agents, and optionally Secondary Additional Therapeutic Agents, exert their prophylactic or therapeutic effect, or vice versa.

In another embodiment, the Compound of Table 1 and the Primary Additional Therapeutic Agents, and optionally Secondary Additional Therapeutic Agents, are administered in doses commonly employed when such agents are used as monotherapy for treating a viral infection.

In another embodiment, the Compound of Table 1 and the Primary Additional Therapeutic Agents, and optionally Secondary Additional Therapeutic Agents, are administered in doses lower than the doses commonly employed when such agents are used as monotherapy for treating a viral infection.

In still another embodiment, the Compound of Table 1 and the Primary Additional Therapeutic Agents, and optionally Secondary Additional Therapeutic Agents, act synergistically and are administered in doses lower than the doses commonly employed when such agents are used as monotherapy for treating a viral infection.

In one embodiment, Compound of Table 1 and the Primary Additional Therapeutic Agents, and optionally Secondary Additional Therapeutic Agents, are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

In one embodiment, the viral infection is HCV infection.

The Compound of Table 1 and the Primary Additional Therapeutic Agents, and optionally Secondary Additional Therapeutic Agents, can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of an HCV Inhibitory Composition may inhibit the resistance of a viral infection to one or more of the components of the composition.

### The Compounds of Table 1

The HICs of the present invention comprise a Compound of Table 1, one or more Primary Additional Therapeutic Agents and optionally, one or more Secondary Additional Therapeutic Agents.

In one embodiment, the Compound of Table 1 is Compound 5, 6, 7 or 12.

In another embodiment, the Compound of Table 1 is Compound 5.

In another embodiment, the Compound of Table 1 is Compound 6.

In still another embodiment, the Compound of Table 1 is Compound 7.

In another embodiment, the Compound of Table 1 is Compound 12.

### Primary Additional Therapeutic Agents

Primary additional therapeutic agents useful in the present compositions and methods include HCV protease inhibitors, HCV polymerase inhibitors, HCV NS4A inhibitors and HCV NS5A inhibitors and pharmaceutically acceptable salts thereof, such that the HCV protease inhibitors are not any of the Compounds of Table 1.

HCV protease inhibitors useful as Primary Additional Therapeutic Agents in the present compositions and methods include, but are not limited to, those disclosed in U.S. Pat. Nos. 7,494,988, 7,485,625, 7,449,447, 7,442,695, 7,425,576, 7,342,041, 7,253,160, 7,244,721, 7,205,330, 7,192,957, 7,186,747, 7,173,057, 7,169,760, 7,012,066, 6,914,122, 6,911,428, 6,894,072, 6,846,802, 6,838,475, 6,800,434, 6,767,991, 5,017,380, 4,933,443, 4,812,561 and 4,634,697; U.S. Patent Publication Nos. US20020068702, US20020160962, US20050119168, US20050176648, US20050209164, US20050249702 and US20070042968; and International Publication Nos. WO 03/006490, WO 03/087092, WO 04/092161 and WO 08/124148.

Additional HCV protease inhibitors useful as Primary Additional Therapeutic Agents in the present compositions and methods include, but are not limited to, VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott/Enanta), TMC-435350 (Medivir), RG7227 (Danoprevir, InterMune/Roche), EA-058 (Abbott/Enanta), EA-063 (Abbott/Enanta), GS-9256 (Gilead), IDX-320 (Idenix), ACH-1625 (Achillion), ACH-2684 (Achillion), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), BMS-650032 (Bristol-Myers Squibb), VX-985 (Vertex) and PHX1766 (Phenomix).

Further examples of HCV protease inhbitors useful as Primary Additional Therapeutic Agents in the present compositions and methods include, but are not limited to, those disclosed in Landro et al., Biochemistry, 36(31):9340-9348 (1997); Ingallinella et al., Biochemistry, 37(25):8906-8914 (1998); Llinás-Brunet et al., Bioorg Med Chem Lett, 8(13):1713-1718 (1998); Martin et al., Biochemistry, 37(33):11459-11468 (1998); Dimasi et al., J Virol, 71(10):7461-7469 (1997); Martin et al., Protein Eng, 10(5):607-614 (1997); Elzouki et al., J Hepat, 27(1):42-48 (1997); Bio World Today, 9(217):4 (Nov. 10, 1998); U.S. Patent Publication Nos. US2005/0249702 and US 2007/0274951; and International Publication Nos. WO 98/14181, WO 98/17679, WO 98/17679, WO 98/22496 and WO 99/07734 and WO 05/087731.

HCV polymerase inhibitors useful as Primary Additional Therapeutic Agents in the present compositions and methods include, but are not limited to, SCH900942 (Schering-Plough), SCH900188 (Schering-Plough), BMS-791325 (Bristol-Myers Squibb), VP-19744 (Wyeth/ViroPharma), PSI-7851 (Pharmasset), RG7128 (Roche/Pharmasset), PSI-7977 (Pharmasset), PSI-938 (Pharmasset), PSI-879 (Pharmasset), PSI-661 (Pharmasset), PF-868554/filibuvir (Pfizer), VCH-759/VX-759 (ViroChem Pharma/Vertex), HCV-371 (Wyeth/VirroPharma), HCV-796 (WyethNiroPharma), IDX-184 (Idenix), IDX-375 (Idenix), NM-283 (Idenix/Novartis), GL-60667 (Genelabs), JTK-109 (Japan Tobacco), PSI-6130 (Pharmasset), R1479 (Roche), R-1626 (Roche), R-7128 (Roche), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), VCH-222/VX-222 (ViroChem/Vertex), VCH-916 (ViroChem), VCH-716(ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews, 1:867 (2002); and Beaulieu et al., Current Opinion in Investigational Drugs, 5:838 (2004).

Other HCV polymerase inhibitors useful as Primary Additional Therapeutic Agents in the present compositions and methods include, but are not limited to, those disclosed in International Publication Nos. WO 08/082484, WO 08/082488, WO 08/083351, WO 08/136815, WO 09/032116, WO 09/032123, WO 09/032124 and WO 09/032125 and compounds T1-T4, depicted below:

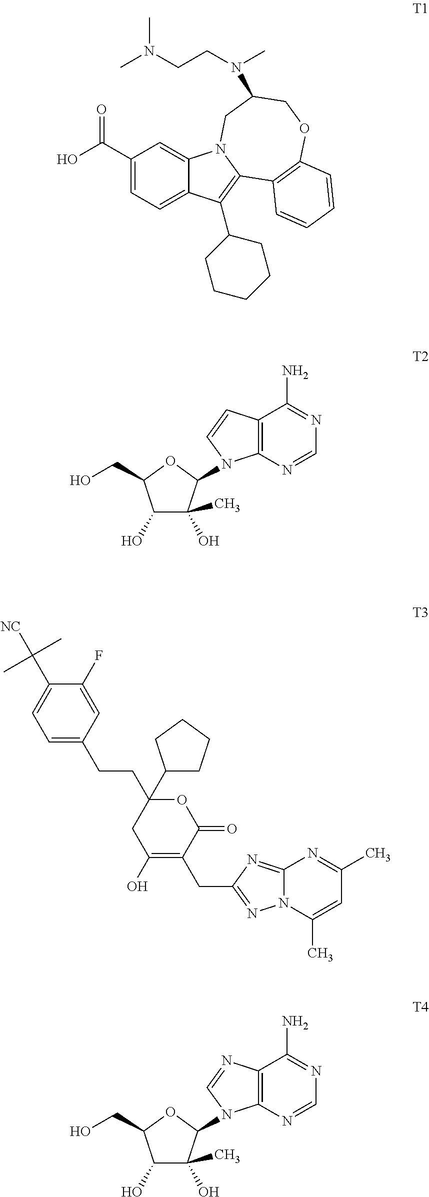 [SMILES: CN(C)CCN(C)[C@H]1COc2ccccc2-c2c(C3CCCCC3)c3ccc(C(=O)O)cc3n2C1 ; C[C@@]1(O)[C@H](O)[C@@H](CO)O[C@H]1n1ccc2c(N)ncnc21 ; Cc1cc(C)n2nc(CC3=C(O)CC(CCc4ccc(C(C)(C)C#N)c(F)c4)(C4CCCC4)OC3=O)nc2n1 ; C[C@@]1(O)[C@H](O)[C@@H](CO)O[C@H]1n1cnc2c(N)ncnc21]

In one embodiment, the HCV polymerase inhibitor is:

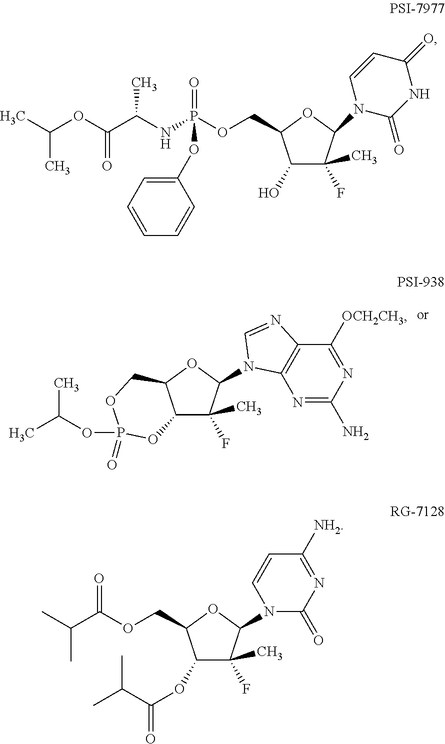

In a preferred embodiment, the HCV polymerase inhibitor is PSI-7977.

HCV NS4A inhibitors useful as Primary Additional Therapeutic Agents in the present compositions and methods include, but are not limited to, those disclosed in U.S. Pat. Nos. 7,476,686 and 7,273,885; U.S. Patent Publication No. US20090022688; and International Publication Nos. WO 2006/019831 and WO 2006/019832. Additional HCV NS4A inhibitors useful as Primary Additional Therapeutic Agents in the present compositions and methods include, but are not limited to, AZD2836 (Astra Zeneca), ACH-1095 (Achillion) and ACH-806 (Achillion).

HCV NS5A inhibitors useful as Primary Additional Therapeutic Agents in the present compositions and methods include, but are not limited to, A-832 (Arrow Therapeutics), PPI-461 (Presidio), PPI-1301 (Presidio), BMS-790052 (Bristol-Myers Squibb), BMS-824393 (Bristol-Myers Squibb), ACH-2928 (Achillon) and AZD-7295 (Astra Zeneca).

In one embodiment, the HCV NS5A inhibitor is BMS-790052 or PPI-461.

In another embodiment, the HCV NS5A inhibitor is

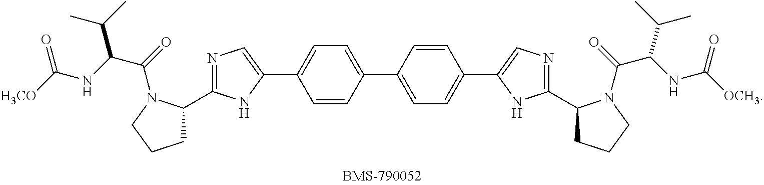 [SMILES: COC(=O)N[C@H](C(=O)N1CCC[C@H]1c1ncc(-c2ccc(-c3ccc(-c4cnc([C@@H]5CCCN5C(=O)[C@@H](NC(=O)OC)C(C)C)[nH]4)cc3)cc2)[nH]1)C(C)C]

In one embodiment, the one or more Primary Additional Therapeutic Agents comprise an HCV protease inhibitor.

In another embodiment, the one or more Primary Additional Therapeutic Agents comprise an HCV polymerase inhibitor.

In another embodiment, the one or more Primary Additional Therapeutic Agents comprise a nucleoside HCV polymerase inhibitor.

In still another embodiment, the one or more Primary Additional Therapeutic Agents comprise a non-nucleoside HCV polymerase inhibitor.

In another embodiment, the one or more Primary Additional Therapeutic Agents comprise an HCV NS5A inhibitor.

In yet another embodiment, the one or more Primary Additional Therapeutic Agents comprise an HCV polymerase inhibitor and an HCV NS5A inhibitor.

In another embodiment, the one or more Primary Additional Therapeutic Agents comprise a nucleoside HCV polymerase inhibitor and an HCV NS5A inhibitor.

In a further embodiment, the one or more Primary Additional Therapeutic Agents comprise a non-nucleoside HCV polymerase inhibitor and an HCV NS5A inhibitor.

In another embodiment, the one or more Primary Additional Therapeutic Agents comprise a non-nucleoside HCV polymerase inhibitor and a nucleoside HCV polymerase inhibitor.

In another embodiment, the one or more Primary Additional Therapeutic Agents comprise one or two compounds selected from PSI-7977, RG-7128, PSI-938, BMS-790052 or PPI-461.

In another embodiment, the one or more Primary Additional Therapeutic Agents comprise: (i) PSI-7977 or PSI-938 and (ii) BMS-790052.

In still another embodiment, the one or more Primary Additional Therapeutic Agents comprise PSI-7977 and BMS-790052.

### Secondary Additional Therapeutic Agents

The present invention also provides HCV Inhibitory Compositions comprising: (i) a Compound of Table 1 or a pharmaceutically acceptable salt thereof; (ii) one or more Primary Additional Therapeutic Agents or a pharmaceutically acceptable salt thereof and (iii) one or more Secondary Additional Therapeutic Agents or a pharmaceutically acceptable salt thereof methods of use thereof.

Secondary Additional Therapeutic Agents useful in the present compositions and methods include, but are not limited to, an interferon, an immunomodulator, a viral replication inhibitor, an antisense agent, a viral helicase inhibitor, a virion production inhibitor, a viral entry inhibitor, a viral assembly inhibitor and an antibody therapy (monoclonal or polyclonal), such that the Secondary Additional Therapeutic Agent is neither a Compound of Table 1 or a Primary Additional Therapeutic Agent.

Interferons useful as Secondary Additional Therapeutic Agents in the present compositions and methods include, but are not limited to, interferon alfa-2a, interferon alfa-2b, interferon alfacon-1 and PEG-interferon alpha conjugates. “PEG-interferon alpha conjugates” are interferon alpha molecules covalently attached to a PEG molecule. Illustrative PEG-interferon alpha conjugates include interferon alpha-2a (ROFERON™, Hoffman La-Roche, Nutley, N.J.) in the form of pegylated interferon alpha-2a (e.g., as sold under the trade name PEGASYS™), interferon alpha-2b (Intron™, from Schering-Plough Corporation) in the form of pegylated interferon alpha-2b (e.g., as sold under the trade name PEG-INTRON™ from Schering-Plough Corporation), interferon alpha-2b-XL (e.g., as sold under the trade name PEG-INTRON™), interferon alpha-2c (BEROFOR ALPHA™, Boehringer Ingelheim, Ingelheim, Germany), PEG-interferon lambda (Bristol-Myers Squibb and ZymoGenetics), interferon alfa-2b alpha fusion polypeptides, interferon fused with the human blood protein albumin (ALBUFERON™, Human Genome Sciences), Omega Interferon (Intarcia), Locteron controlled release interferon (Biolex/OctoPlus), Biomed-510 (omega interferon), Peg-IL-29 (ZymoGenetics), Locteron CR (Octoplus), R-7025 (Roche), IFN-α-2b-XL (Flamel Technologies), belerofon (Nautilus) and consensus interferon as defined by determination of a consensus sequence of naturally occurring interferon alphas (INFERGEN™, Amgen, Thousand Oaks, Calif.).

Antibody therapy agents useful as Secondary Additional Therapeutic Agents in the present compositions and methods include, but are not limited to, antibodies specific to IL-10 (such as those disclosed in US Patent Publication No. US2005/0101770, humanized 12G8, a humanized monoclonal antibody against human IL-10, plasmids containing the nucleic acids encoding the humanized 12G8 light and heavy chains were deposited with the American Type Culture Collection (ATCC) as deposit numbers PTA-5923 and PTA-5922, respectively), and the like).

Viral replication inhibitors useful as Secondary Additional Therapeutic Agents in the present compositions and methods include, but are not limited to, HCV replicase inhibitors, IRES inhibitors, NS3 helicase inhibitors, ribavirin, AZD-2836 (Astra Zeneca), viramidine, A-831 (Arrow Therapeutics), EDP-239 (Enanta), ACH-2928 (Achillion), GS-5885 (Gilead); an antisense agent or a therapeutic vaccine.

Viral entry inhibitors useful as Secondary Additional Therapeutic Agents in the present compositions and methods include, but are not limited to, PRO-206 (Progenies), REP-9C (REPICor), SP-30 (Samaritan Pharmaceuticals) and ITX-5061 (iTherx).

HCV replicase inhibitors useful as Secondary Additional Therapeutic Agents in the present compositions and methods include, but are not limited to, those disclosed in U.S. Patent Publication No. US20090081636.

Therapeutic vaccines useful as Secondary Additional Therapeutic Agents in the present compositions and methods include, but are not limited to, IC41 (Intercell Novartis), CSL123 (Chiron/CSL), GI 5005 (Globeimmune), TG-4040 (Transgene), GNI-103 (GENimmune), Hepavaxx C (ViRex Medical), ChronVac-C(Inovio/Tripep), PeviPRO™ (Pevion Biotect), HCV/MF59 (Chiron/Novartis), MBL-HCV 1 (MassBiologics), GI-5005 (GlobeImmune), CT-011 (CureTech/Teva) and Civacir (NABI).

Examples of further agents useful as Secondary Additional Therapeutic Agents in the present compositions and methods include, but are not limited to, Ritonavir (Abbott), TT033 (Benitec/Tacere Bio/Pfizer), Sirna-034 (Sirna Therapeutics), GNI-104 (GENimmune), GI-5005 (GlobeImmune), IDX-102 (Idenix), LEVOVIRIN™ (ICN Pharmaceuticals, Costa Mesa, Calif.); Humax (Genmab), ITX-2155 (Ithrex/Novartis), PRO 206 (Progenies), HepaCide-I (NanoVirocides), MX3235 (Migenix), SCY-635 (Scynexis); KPE02003002 (Kemin Pharma), Lenocta (VioQuest Pharmaceuticals), IET—Interferon Enhancing Therapy (Transition Therapeutics), Zadaxin (SciClone Pharma), VP 50406™ (Viropharma, Incorporated, Exton, Pa.); Taribavirin (Valeant Pharmaceuticals); Nitazoxanide (Romark); Debio 025 (Debiopharm); GS-9450 (Gilead); PF-4878691 (Pfizer); ANA773 (Anadys); SCV-07 (SciClone Pharmaceuticals); NIM-881 (Novartis); ISIS 14803™ (ISIS Pharmaceuticals, Carlsbad, Calif.); HEPTAZYME™ (Ribozyme Pharmaceuticals, Boulder, Colo.); THYMOSIN™ (SciClone Pharmaceuticals, San Mateo, Calif.); MAXAMINE™ (Maxim Pharmaceuticals, San Diego, Calif.); NKB-122 (JenKen Bioscience Inc., North Carolina); Alinia (Romark Laboratories), INFORM-1 (a combination of R7128 and ITMN-191); and mycophenolate mofetil (Hoffman-LaRoche, Nutley, N.J.).

In one embodiment, the one or more Secondary Additional Therapeutic Agents are selected from an interferon and ribavirin.

In another embodiment, the one or more Secondary Additional Therapeutic Agents are selected from ribavirin and pegylated interferon-α.

The doses and dosage regimen of the other agents used in the combination therapies of the present invention for the treatment or prevention of HCV infection can be determined by the attending clinician, taking into consideration the approved doses and dosage regimen in the package insert; the age, sex and general health of the patient; and the type and severity of the viral infection or related disease or disorder. When administered in combination, the components of the HCV Inhibitory Composition and the optional Secondary Additional Therapeutic Agents can be administered simultaneously (i.e., in the same composition or in separate compositions one right after the other) or sequentially. This particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms is therefore advantageous.

Generally, a total daily dosage of the components of the HCV Inhibitory Compositions alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

In one embodiment, when the Secondary Additional Therapeutic Agent is INTRON-A interferon alpha 2b (commercially available from Schering-Plough Corp.), this agent is administered by subcutaneous injection at 3 MIU (12 mcg)/0.5 mL/TIW for 24 weeks or 48 weeks for first time treatment.

In another embodiment, when the Secondary Additional Therapeutic Agent is PEG-INTRON interferon alpha 2b pegylated (commercially available from Schering-Plough Corp.), this agent is administered by subcutaneous injection at 1.5 mcg/kg/week, within a range of 40 to 150 mcg/week, for at least 24 weeks.

In another embodiment, when the Secondary Additional Therapeutic Agent is ROFERON A interferon alpha 2a (commercially available from Hoffmann-La Roche), this agent is administered by subcutaneous or intramuscular injection at 3 MIU (11.1 mcg/mL)/TIW for at least 48 to 52 weeks, or alternatively 6 MIU/TIW for 12 weeks followed by 3 MIU/TIW for 36 weeks.

In still another embodiment, when the Secondary Additional Therapeutic Agent is PEGASUS interferon alpha 2a pegylated (commercially available from Hoffmann-La Roche), this agent is administered by subcutaneous injection at 180 mcg/lmL or 180 mcg/0.5 mL, once a week for at least 24 weeks.

In yet another embodiment, when the Secondary Additional Therapeutic Agent is INFERGEN interferon alphacon-1 (commercially available from Amgen), this agent is administered by subcutaneous injection at 9 mcg/TIW is 24 weeks for first time treatment and up to 15 mcg/TIW for 24 weeks for non-responsive or relapse treatment. In a further embodiment, when the Secondary Additional Therapeutic Agent is

Ribavirin (commercially available as REBETOL ribavirin from Schering-Plough or COPEGUS ribavirin from Hoffmann-La Roche), this agent is administered at a daily dosage of from about 600 to about 1400 mg/day for at least 24 weeks.

In one embodiment, the Secondary Additional Therapeutic Agent is an interferon.

In another embodiment, the Secondary Additional Therapeutic Agent is an immunomodulator.

In another embodiment, the Secondary Additional Therapeutic Agent is a viral replication inhibitor.

In still another embodiment, the Secondary Additional Therapeutic Agent is an antisense agent.

In another embodiment, the Secondary Additional Therapeutic Agent is a viral helicase inhibitor.

In yet another embodiment, the Secondary Additional Therapeutic Agent is a virion production inhibitor.

In another embodiment, the Secondary Additional Therapeutic Agent is a viral entry inhibitor.

In a further embodiment, the Secondary Additional Therapeutic Agent is a viral assembly inhibitor.

In another embodiment, the Secondary Additional Therapeutic Agent is an antibody therapy (monoclonal or polyclonal).

In another embodiment, the Secondary Additional Therapeutic Agent is an HCV NS2 inhibitor.

In still another embodiment, the Secondary Additional Therapeutic Agent is an HCV NS4B inhibitor.

In another embodiment, the Secondary Additional Therapeutic Agent is an HCV NS3 helicase inhibitor.

In another embodiment, the Secondary Additional Therapeutic Agent is an HCV IRES inhibitor.

In a further another embodiment, the Secondary Additional Therapeutic Agent is an HCV p7 inhibitor.

In one embodiment, the HCV Inhibitory Compositions comprise: (i) one of Compounds 5, 6, 7 and 12 and (ii) an HCV polymerase inhibitor.

In another embodiment, the HCV Inhibitory Compositions comprise: (i) one of Compounds 5, 6, 7 and 12 and (ii) an HCV NS5A inhibitor.

In another embodiment, the HCV Inhibitory Compositions comprise: (i) one of Compounds 5, 6, 7 and 12 and (ii) an HCV NS5A inhibitor; and (iii) an HCV polymerase inhibitor.

In another embodiment, the HCV Inhibitory Compositions comprise: (i) one of Compounds 5, 6, 7 and 12 and (ii) one of PSI-7977, PSI-938 and RG7128.

In another embodiment, the HCV Inhibitory Compositions comprise: (i) one of Compounds 5, 6, 7 and 12 and (ii) one of PSI-7977, PSI-938 and RG7128; and (iii) an HCV NS5A inhibitor.

In another embodiment, the HCV Inhibitory Compositions comprise: (i) one of Compounds 5, 6, 7 and 12 and (ii) one of PSI-7977, PSI-938 and RG7128; and (iii) BMS790052.

In one embodiment, the HCV Inhibitory Compositions comprise: (i) one of Compounds 5, 6, 7 and 12; (ii) an HCV polymerase inhibitor; and (iii) one or both or pegylated interferon-α and ribavirin.

In another embodiment, the HCV Inhibitory Compositions comprise: (i) one of Compounds 5, 6, 7 and 12 and (ii) an HCV NS5A inhibitor; and (iii) one or both or pegylated interferon-α and ribavirin.

In another embodiment, the HCV Inhibitory Compositions comprise: (i) one of Compounds 5, 6, 7 and 12 and (ii) an HCV NS5A inhibitor; (iii) an HCV polymerase inhibitor; (iv) one or both or pegylated interferon-α and ribavirin.

In another embodiment, the HCV Inhibitory Compositions comprise: (i) one of Compounds 5, 6, 7 and 12; (ii) one of PSI-7977, PSI-938 and RG7128; and (iii) one or both or pegylated interferon-α and ribavirin.

In another embodiment, the HCV Inhibitory Compositions comprise: (i) one of Compounds 5, 6, 7 and 12 and (ii) one of PSI-7977, PSI-938 and RG7128; (iii) an HCV NS5A inhibitor; and (iv) one or both or pegylated interferon-α and ribavirin.

In another embodiment, the HCV Inhibitory Compositions comprise: (i) one of Compounds 5, 6, 7 and 12 and (ii) one of PSI-7977, PSI-938 and RG7128; (iii) BMS790052; (iv) one or both or pegylated interferon-α and ribavirin.

In one embodiment, the HCV Inhibitory Compositions comprise: (i) Compound 5 and (ii) an HCV polymerase inhibitor.

In another embodiment, the HCV Inhibitory Compositions comprise: (i) Compound 5 and (ii) an HCV NS5A inhibitor.

In another embodiment, the HCV Inhibitory Compositions comprise: (i) Compound 5 and (ii) an HCV NS5A inhibitor; and (iii) an HCV polymerase inhibitor.

In another embodiment, the HCV Inhibitory Compositions comprise: (i) Compound 5 and (ii) one of PSI-7977, PSI-938 and RG7128.

In another embodiment, the HCV Inhibitory Compositions comprise: (i) Compound 5 and (ii) one of PSI-7977, PSI-938 and RG7128; and (iii) an HCV NS5A inhibitor.

In another embodiment, the HCV Inhibitory Compositions comprise: (i) Compound 5 and (ii) one of PSI-7977, PSI-938 and RG7128; and (iii) BMS790052.

In one embodiment, the HCV Inhibitory Compositions comprise: (i) Compound 5; (ii) an HCV polymerase inhibitor; and (iii) one or both or pegylated interferon-α and ribavirin.

In another embodiment, the HCV Inhibitory Compositions comprise: (i) Compound 5 and (ii) an HCV NS5A inhibitor; and (iii) one or both or pegylated interferon-α and ribavirin.

In another embodiment, the HCV Inhibitory Compositions comprise: (i) Compound 5 and (ii) an HCV NS5A inhibitor; (iii) an HCV polymerase inhibitor; (iv) one or both or pegylated interferon-α and ribavirin.

In another embodiment, the HCV Inhibitory Compositions comprise: (i) Compound 5; (ii) one of PSI-7977, PSI-938 and RG7128; and (iii) one or both or pegylated interferon-α and ribavirin.

In another embodiment, the HCV Inhibitory Compositions comprise: (i) Compound 5 and (ii) one of PSI-7977, PSI-938 and RG7128; (iii) an HCV NS5A inhibitor; and (iv) one or both or pegylated interferon-α and ribavirin.

In another embodiment, the HCV Inhibitory Compositions comprise: (i) Compound 5 and (ii) one of PSI-7977, PSI-938 and RG7128; (iii) BMS790052; (iv) one or both or pegylated interferon-α and ribavirin.

### Compositions and Administration

Due to their activity, the HCV Inhibitory Compositions are useful in veterinary and human medicine. As described above, the HCV Inhibitory Compositions are useful for treating or preventing HCV infection in a patient in need thereof.

In the compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Aerosol preparations suitable for inhalation may include solutions and solids in powder form, which may be in combination with a pharmaceutically acceptable carrier, such as an inert compressed gas.

Also included are solid form preparations which are intended to be converted, shortly before use, to liquid form preparations for either oral or parenteral administration. Such liquid forms include solutions, suspensions and emulsions

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, one or more of the components of the HCV Inhibitory Compositions are administered orally.

In another embodiment, one or more of the components of the HCV Inhibitory Compositions are administered intravenously.

In still another embodiment, one or more of the components of HCV Inhibitory Compositions are administered sublingually.

In one embodiment, a pharmaceutical preparation comprising the entire HCV Inhibitory Composition is in unit dosage form. In such form, the preparation is subdivided into unit doses containing effective amounts of the active components.

In another embodiment, separate pharmaceutical preparations, each containing a component of the HCV Inhibitory Composition is in unit dosage form. In such form, the each of the preparation is subdivided into unit doses containing effective amounts of the active components.

The HCV Inhibitory Compositions can be prepared according to conventional mixing, granulating or coating methods, respectively, and the present compositions can contain, in one embodiment, from about 0.1% to about 99% of the HCV Inhibitory Composition(s) by weight or volume. In various embodiments, the present compositions can contain, in one embodiment, from about 1% to about 70% or from about 5% to about 60% of the active components by weight or volume.

The quantity of each component of an HCV Inhibitory Composition in a unit dose of preparation may be varied or adjusted from about 1 mg to about 2500 mg. In various embodiments, the quantity is from about 10 mg to about 1000 mg, 1 mg to about 500 mg, 1 mg to about 100 mg, and 1 mg to about 100 mg.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The amount and frequency of administration of the HCV Inhibitory Compositions will be regulated according to the judgment of the attending clinician considering such factors as age, condition and size of the patient as well as severity of the symptoms being treated. Generally, a total daily dosage of the HCV Inhibitory Compositions range from about 0.1 to about 2000 mg per day, although variations will necessarily occur depending on the target of therapy, the patient and the route of administration. In one embodiment, the dosage is from about 1 to about 200 mg/day, administered in a single dose or in 2-4 divided doses. In another embodiment, the dosage is from about 10 to about 2000 mg/day, administered in a single dose or in 2-4 divided doses. In another embodiment, the dosage is from about 100 to about 2000 mg/day, administered in a single dose or in 2-4 divided doses. In still another embodiment, the dosage is from about 500 to about 2000 mg/day, administered in a single dose or in 2-4 divided doses.

### Kits

In one aspect, the present invention provides a kit comprising an HCV, wherein all of the components of the HCV are present in the same container, wherein the amounts of the active ingredients together result in a desired therapeutic effect.

In another aspect, the present invention provides a kit comprising an HCV, wherein the components of the HCV are provided in two or more separate containers, wherein the amounts of the three active ingredients together result in a desired therapeutic effect.

In another aspect, the present invention provides a kit comprising an HCV, wherein the Compound of Table 1 and the one or more Primary Additional Therapeutic Agents are each provided in a separate container, wherein the amounts of the three active ingredients together result in a desired therapeutic effect.

In still another aspect, the present invention provides a kit comprising an HCV, wherein the Compound of Table 1, the one or more Primary Additional Therapeutic Agents and the one or more Secondary Additional Therapeutic Agents are each provided in a separate container, wherein the amounts of the three active ingredients together result in a desired therapeutic effect.

### EXAMPLES

### Example 1

### Preparation of the Compounds of Table 1

The Compounds of Table 1 can be made, for example, using the methods described in U.S. Patent Publication No. US 2010/0099695 and U.S. Pat. Nos. 7,012,066, 7,244,721, 7,470,664 and 7,973,040, each of which are incorporated herein by reference in their entirety.

Compound T1 can be made as described in U.S. Pat. No. 7,662,809.

Compound T2 and T4 can be made as described in U.S. Pat. No. 7,105,499, which is incorporated by reference herein in its entirety.

Compound T3 can be made using methods described in International Publication Nos. WO 2006018725 and WO 2004074270 and in US Patent Publication No. US 2005176701, each of which is incorporated by reference herein in its entirety.

The other Primary Additional Therapeutic Agents and Secondary Additional Therapeutic Agents are well-known therapeutic compounds and references describing their synthesis are publicly available and will be easily found by the skilled artisan.

### Example 2

### Assay for Inhibition of HCV RNA Replication

Compounds were evaluated for their ability to affect the replication of Hepatitis C Virus RNA in cultured hepatoma (HuH-7) cells containing a subgenomic HCV Replicon. The details of the assay are described below. This Replicon assay is a modification of that described in V. Lohmann et al., “Replication of a Sub-genomic Hepatitis C Virus RNAs in a Hepatoma Cell Line,” Science 285:110 (1999).

### Protocol:

The assay used is an in situ Ribonuclease protection, Scintillation Proximity based-plate assay (SPA). 10,000-40,000 cells were plated in 100-200 μL of media containing 0.8 mg/mL G418 in 96-well CYTOSTAR plates (Amersham). Compounds were added to cells at various concentrations up to 100 μM in 1% DMSO at time 0 to 18 h and then cultured for 24-96 h. Cells were fixed (20 min, 10% formalin), permeabilized (20 min, 0.25% TRITON X-100/PBS) and hybridized (overnight, 50° C.) with a single-stranded ³³P RNA probe complementary to the (+) strand NS5B (or other genes) contained in the RNA viral genome. Cells were washed, treated with RNAse, washed, heated to 65° C. and counted in a Top-Count. Inhibition of replication was read as a decrease in counts per minute (cpm).

Human HuH-7 hepatoma cells, which were selected to contain a subgenomic replicon, carry a cytoplasmic RNA consisting of an HCV 5′ non-translated region (NTR), a neomycin selectable marker, an EMCV IRES (internal ribosome entry site), and HCV non-structural proteins NS3 through NS5B, followed by the 3′ NTR.

### Example 3

### Evaluation of Inhibition of the HCV Subgenomic Replicon by Combinations of Compounds

The combined antiviral effects of two compounds tested in combination are evaluated in the HCV replicon assay. Each compound is assessed for its ability to inhibit viral replication individually and in combinations with the other compound.

The combined antiviral effect of two compounds is evaluated using an analytical computer program (MACSYNERGY™ II) based on the Bliss independence model (Bliss, C. I.: The toxicity of poisons applied jointly. Ann. Appl. Biol. 26: 585-615, 1939) that assumes independent action of each of the compounds to inhibit viral replication to calculate the predicted additive level of inhibition of the combination. The experimentally determined fraction of inhibition is then compared to the predicted additive level across the range of compound concentrations included in the combinations. Greater than predicted inhibition appears on the graph as a surface above the plane of additivity. Alternately, another method of analysis has been employed that uses a similar response additive model assuming independent action for each of the compounds to predict the level of inhibition expected for additivity (Barton, C N, Braunberg, R C, and Friedman, L. Nonlinear statistical models for the joint action of toxins. Biometrics 1993 49, 95-105). The experimentally determined fraction of inhibition for each pairwise combination is then compared to the predicted additive level and assigned Interference when the inhibition in the combination is below the inhibition by either compound alone; Inert when the inhibition in the combination is equal to the inhibition by the more inhibitory of the compounds when tested alone; Subadditive when the inhibition by the combination is greater than the inhibition of either compound when tested alone but below the predicted inhibition assuming additivity; Additive when the inhibition by the combination is equal to the predicted inhibition for additivity; Synergistic when the inhibition by the combination is greater than the inhibition predicted for additivity; or saturated when the inhibition by the combination is close to 100%.

### Example 4

### Evaluation of the Inhibition of HCV Subgenomic Replicon Variants with Decreased Susceptibility to Inhibition by a Protease Inhibitor

There have been disclosures of HCV replicons containing amino acid changes that cause reduced susceptibility to inhibition by a class of known inhibitory compounds such as compounds that inhibit HCV NS3/4A protease activity (Courcambeck, et al., Resistance of hepatitis C virus to NS3-4A protease inhibitors: mechanisms of drug resistance induced by R155Q, A156T, D168A and D168V mutations. Antiviral Therapy 2006, 11, 847-55). Typically the amino acid variations that cause reduced susceptibility to inhibition occur in the viral enzyme that is inhibited by the compound. Generally, the amino acid variations cause reduced susceptibility to inhibition by causing reduced binding of the compound to the target enzyme. One advantage to the use of combinations of two or more compounds that effect viral replication by different mechanisms in a combination to treat HCV infection is that the amino acid variations that cause reduced susceptibility to inhibition brought about by one class of compound may have little or no effect on the ability of the second class of compound to inhibit viral replication.

### Example 5

### Replicon Assay Method

The day prior to titration replicon cells are seeded into a 96-well Costar black-walled clear bottom plate (3,000 cells/well) in 100 μl complete D-MEM and incubated 0/N at 37° C. The next morning 25 μl of compound dilutions (5× final concentration) in complete DMEM and 5% DMSO are added, adjusting the final DMSO concentration to 1.0%. Cells with compound are incubated for 48 hours at 37° C.

Total RNA is isolated from cells of each well using the QIAGEN RNeasy 96-well kit according to manufacturer's instructions (QIAGEN, Inc. Valencia, Calif.). RNA is eluted twice in 60 μl aliquots and combined. TaqMan reactions are performed with TaqMan EZ RT-PCR (Applied Biosystems Inc., Foster City, Calif.) using 5 μl RNA in a final reaction volume of 25 Cycling conditions are: 50° C. 2 minutes, 60° C. 30 minutes, 95° C. 5 minutes, followed by 40 cycles of 94° C. 20 seconds, 55° C. for 1 minute. Reactions are run on an ABI 7500 (Applied Biosystems Inc., Foster City Calif.).

Primers and probes to detect replicon RNA are directed against the co-encoded Neo^{r} gene and are shown:

Forward primer
(SEQ ID NO. 1)
5′ CCG GCT ACC TGC CCA TTC
Reverse primer
(SEQ ID NO. 2)
5′ CCA GAT CAT CCT GAT CGA CAA G
Probe: FAM-
(SEQ ID NO. 3)
ACA TCG CAT CGA GCG AGC ACG TAC-Tamra

The probe is synthesized by Applied Biosystems Inc. (Foster City, Calif.).

The normalized value for the amount of replicon RNA is determined by measuring the experimental cycle threshold C_{T} as described above, determining the amount of RNA against a standard curve of total replicon cell RNA, and normalizing this to the signal produced by the cellular human Cyclophilin A gene using Taqman PDAR Human Cyclophilin (Applied Biosystems Inc., Foster City, Calif.).

### Example 6

### Combination of Compound 6 and Compound T1

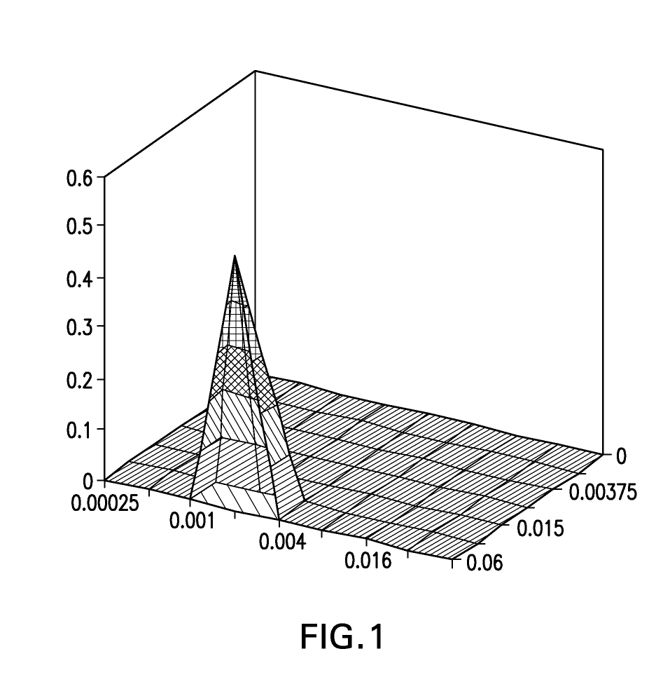

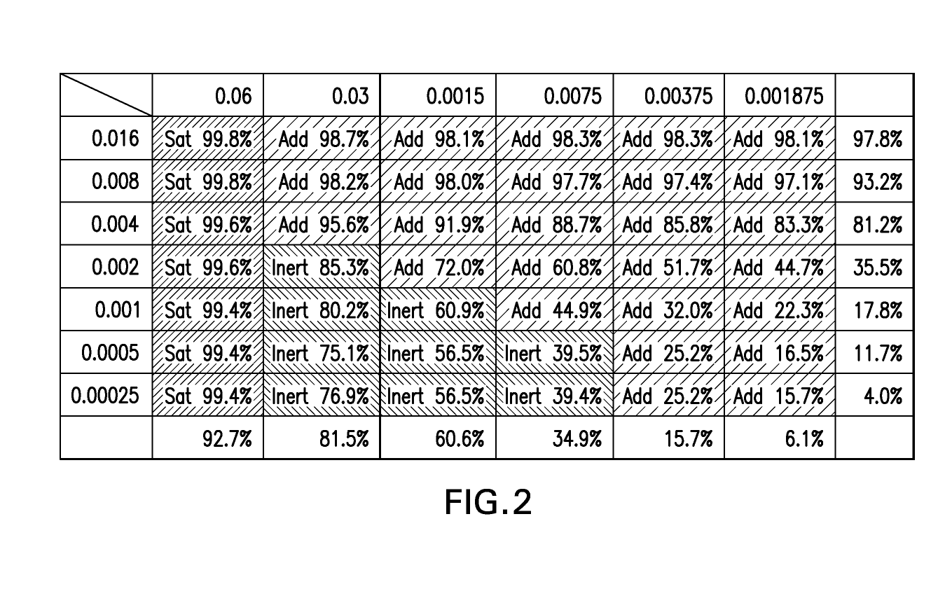

An inhibitor of the HCV NS3/4A serine protease activity, Compound 6, and an inhibitor of the HCV NS5B RNA polymerase activity, Compound T1, were assessed in the HCV replicon assay using HCV genotype 1b (con1) replicon cells. The results from an experiment to determine the combined effects in the HCV replicon assay analyzed using MACSYNERGY™ II are shown in FIG. 1. The results indicate primarily additivity of addition with the volume above the plane of additivity corresponding to a region of synergistic inhibition. The same data were also analyzed using the method of Barton et al., and the results are presented in FIG. 2. The results indicate predominantly additivity of inhibition by the combination in the dose ranges that were tested.

### Example 7

### Combination of Compound 6 and Compound T2

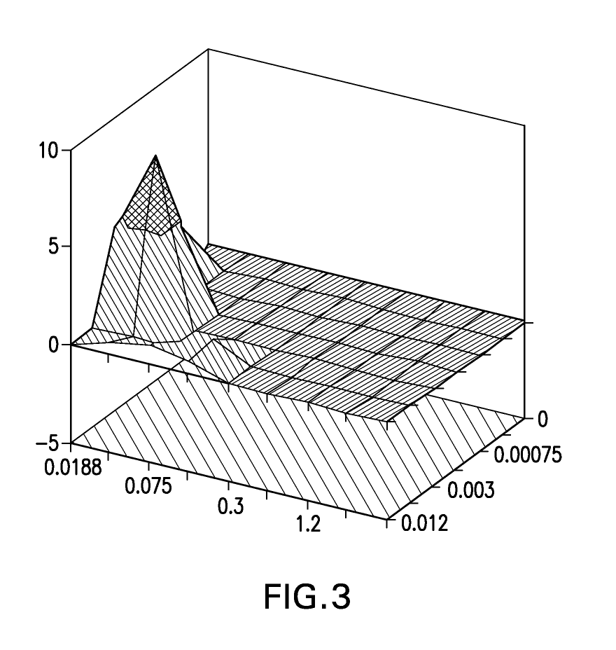

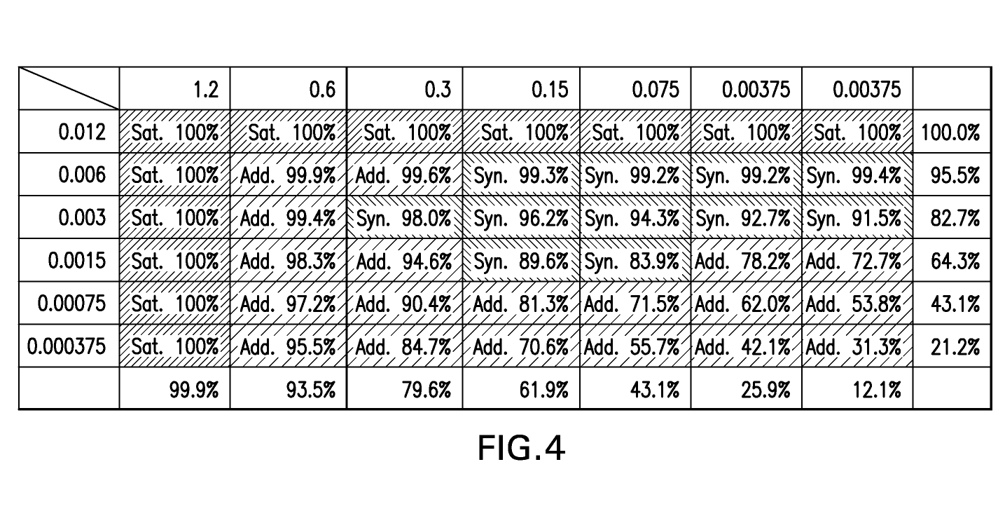

The results from an experiment to determine the combined effects of Compound 6 and Compound T2, in the HCV replicon assay analyzed using MACSYNERGY™ II are shown in FIG. 3. The results indicate a region of synergistic inhibition at low concentrations of Compound T2 and high concentrations of Compound 6. The same data were also analyzed using the method of Barton et al., and the results are presented in FIG. 4. The results indicate synergy and additivity by the combination in the dose ranges that were tested.

### Example 8

### Combination of Compound 6 and Compound T3

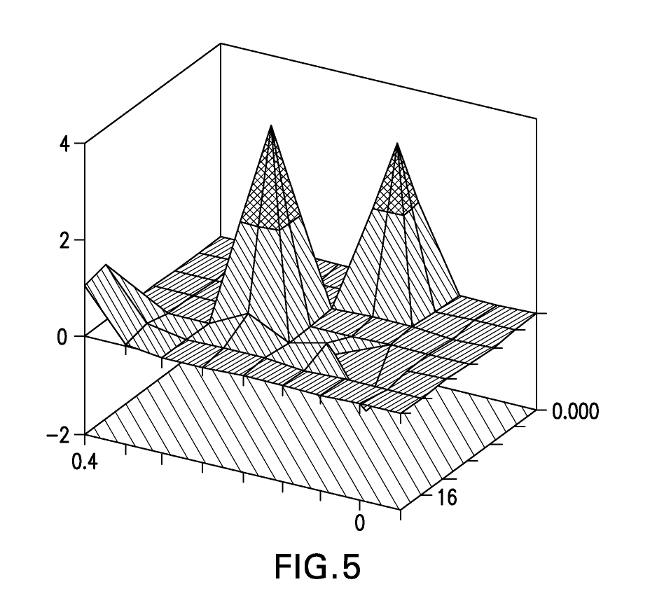

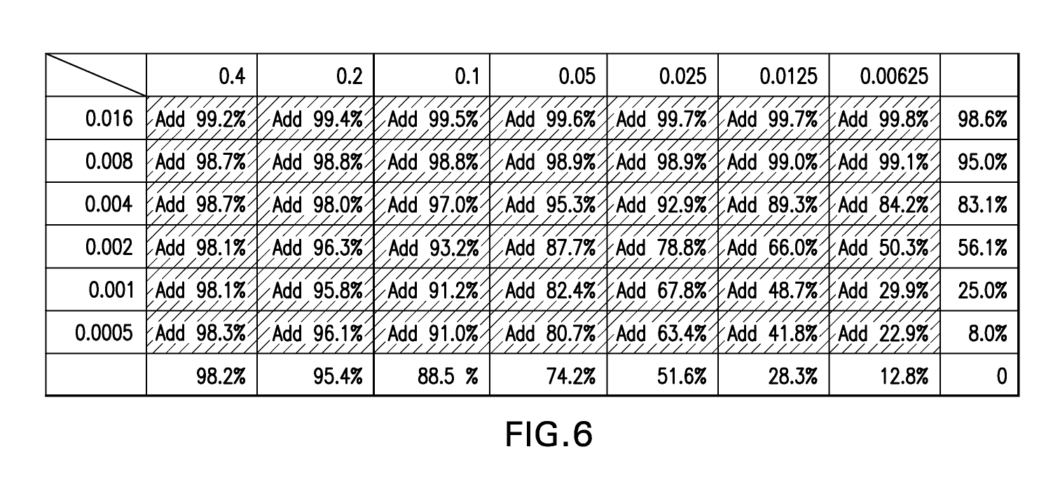

The results from an experiment to determine the combined effects of Compound 6 and Compound T3 and in the HCV replicon assay analyzed using MACSYNERGY™ II are shown in FIG. 5. The results indicate regions of synergistic inhibition. The same data were also analyzed using the method of Barton et al., and the results are presented in FIG. 6. The results indicate additivity by the combination in the dose ranges that were tested.

### Example 9

### Replicon Assay With Genotypes R155, A156T and D168Y

HCV replicons encoding amino acid variations R155K, A156T, and D168Y in the NS3 protease have been constructed using standard techniques in molecular cloning. The susceptibility of these replicon variants to inhibition by Compound 6 and a Compound T4, was assessed in the HCV replicon assay, and the results are shown in Table 2. The results indicate that the amino acid variations are associated with a significantly reduced susceptibility to inhibition caused by Compound 6 but that there is no significant change in susceptibility to inhibition by Compound T4.

TABLE 2

| Test | Replicon Genotype | | | | |
|---|---|---|---|---|---|
| Compound | wt | R155K | A156T | D168V | D168Y |
| Compound 6 | 1.0 | 315 | 153 | 460 | 723 |
| Compound T4 | 474 | 350 | 460 | 375 | 337 |

Inhibition of wildtype HCV replicon of genotype 1b (con1) and several variants encoding mutations resistant to Compound 6 was assessed using the TaqMan assay described in Example 5. The results indicate significant losses of potency for inhibition of the replicon variants by Compound 6 but no loss of potency of inhibition for Compound T4.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A pharmaceutical composition comprising:
(i) a pharmaceutically acceptable carrier; (ii) a compound selected from Table 1:
TABLE 1

| | |
|---|---|
| 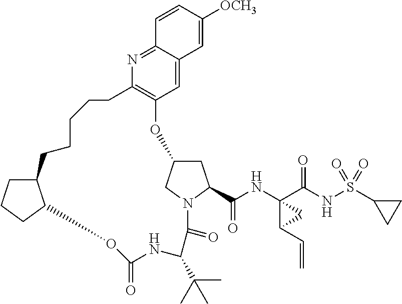 | 1 |
| 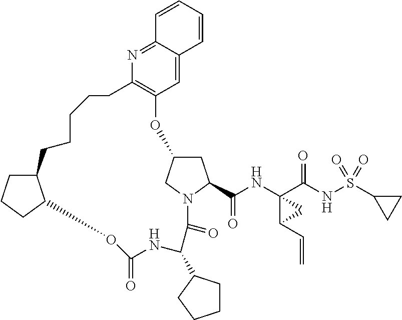 | 2 |
| 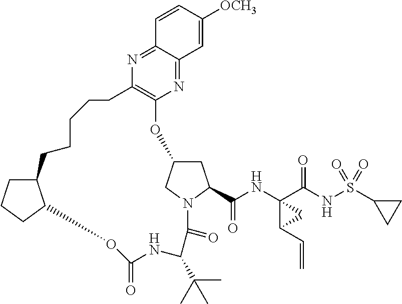 | 3 |
| 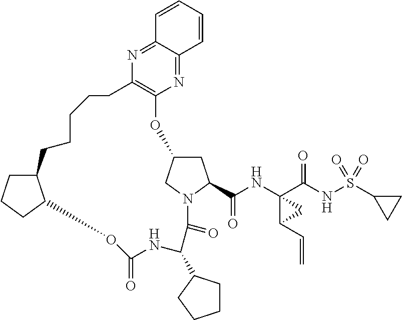 | 4 |
| 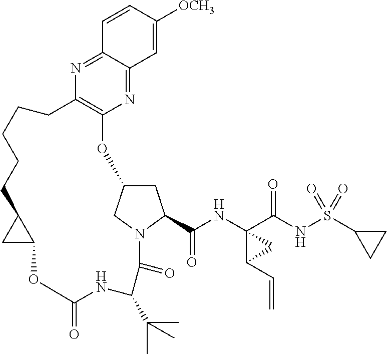 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)NS(=O)(=O)C1CC1] | 5 |
| 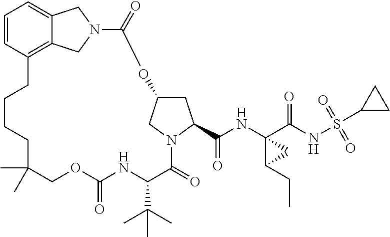 [SMILES: CC[C@@H]1C[C@]1(NC(=O)[C@@H]1C[C@@H]2CN1C(=O)[C@H](C(C)(C)C)NC(=O)OCC(C)(C)CCCCc1cccc3c1CN(C3)C(=O)O2)C(=O)NS(=O)(=O)C1CC1] | 6 |
| 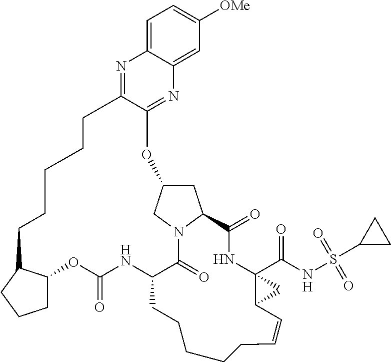 | 7 |
| 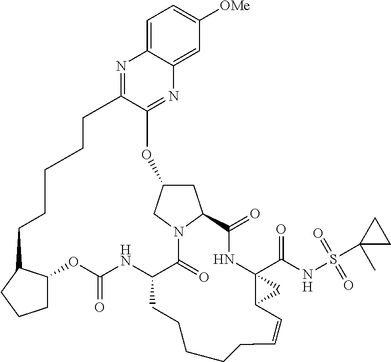 | 8 |
| 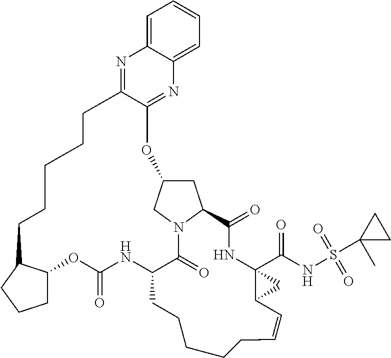 | 9 |
| 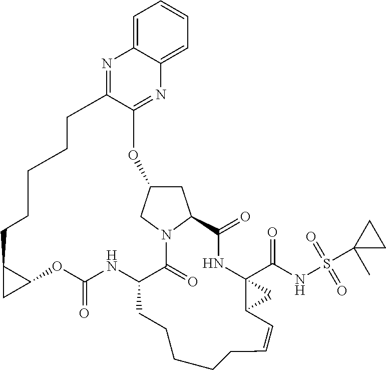 [SMILES: CC1(S(=O)(=O)NC(=O)[C@@]23C[C@H]2/C=C\CCCCC[C@@H]2NC(=O)O[C@@H]4C[C@H]4CCCCCc4nc5ccccc5nc4O[C@@H]4C[C@@H](C(=O)N3)N(C4)C2=O)CC1] | 10 |
| 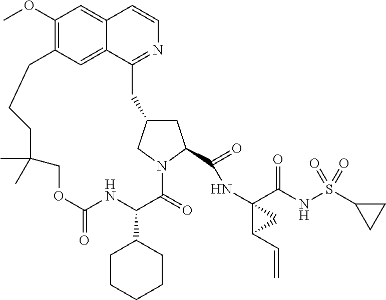 [SMILES: C=C[C@@H]1C[C@]1(NC(=O)[C@@H]1C[C@H]2Cc3nccc4cc(OC)c(cc34)CCCC(C)(C)COC(=O)N[C@@H](C3CCCCC3)C(=O)N1C2)C(=O)NS(=O)(=O)C1CC1] | 11 |
| 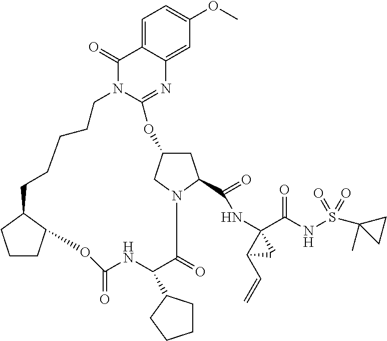 [SMILES: C=C[C@@H]1C[C@]1(NC(=O)[C@@H]1C[C@@H]2CN1C(=O)[C@H](C1CCCC1)NC(=O)O[C@@H]1CCC[C@H]1CCCCCn1c(nc3cc(OC)ccc3c1=O)O2)C(=O)NS(=O)(=O)C1(C)CC1] | 12 |
| 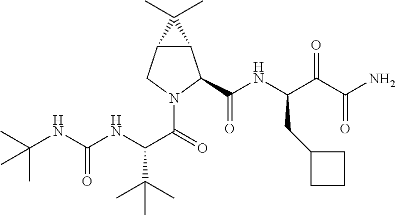 [SMILES: CC(C)(C)NC(=O)N[C@H](C(=O)N1C[C@H]2[C@@H]([C@H]1C(=O)N[C@H](CC1CCC1)C(=O)C(N)=O)C2(C)C)C(C)(C)C] | 13 |
| 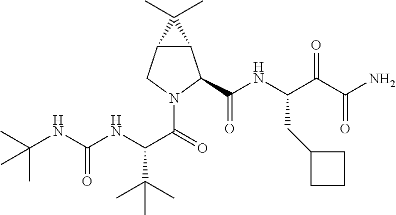 [SMILES: CC(C)(C)NC(=O)N[C@H](C(=O)N1C[C@H]2[C@@H]([C@H]1C(=O)N[C@@H](CC1CCC1)C(=O)C(N)=O)C2(C)C)C(C)(C)C] | 14 |

or a pharmaceutically acceptable salt thereof; and (iii) one or more Primary Additional Therapeutic Agents, or a pharmaceutically acceptable salt thereof, wherein the one or more Primary Additional Therapeutic Agents are selected from the group consisting of HCV protease inhibitors, HCV polymerase inhibitors, HCV NS4A inhibitors and HCV NS5A inhibitors, wherein the one or more Primary Additional Therapeutic Agents do not comprise a Compound of Table 1, and wherein the amounts of the Compound of Table 1 and the one or more Primary Additional Therapeutic Agents are together effective to treat HCV infection in a patient, such that when the one or more Primary Additional Therapeutic Agents comprise an HCV NS5A inhibitor, the HCV NS5A inhibitor is not one of the following compounds: 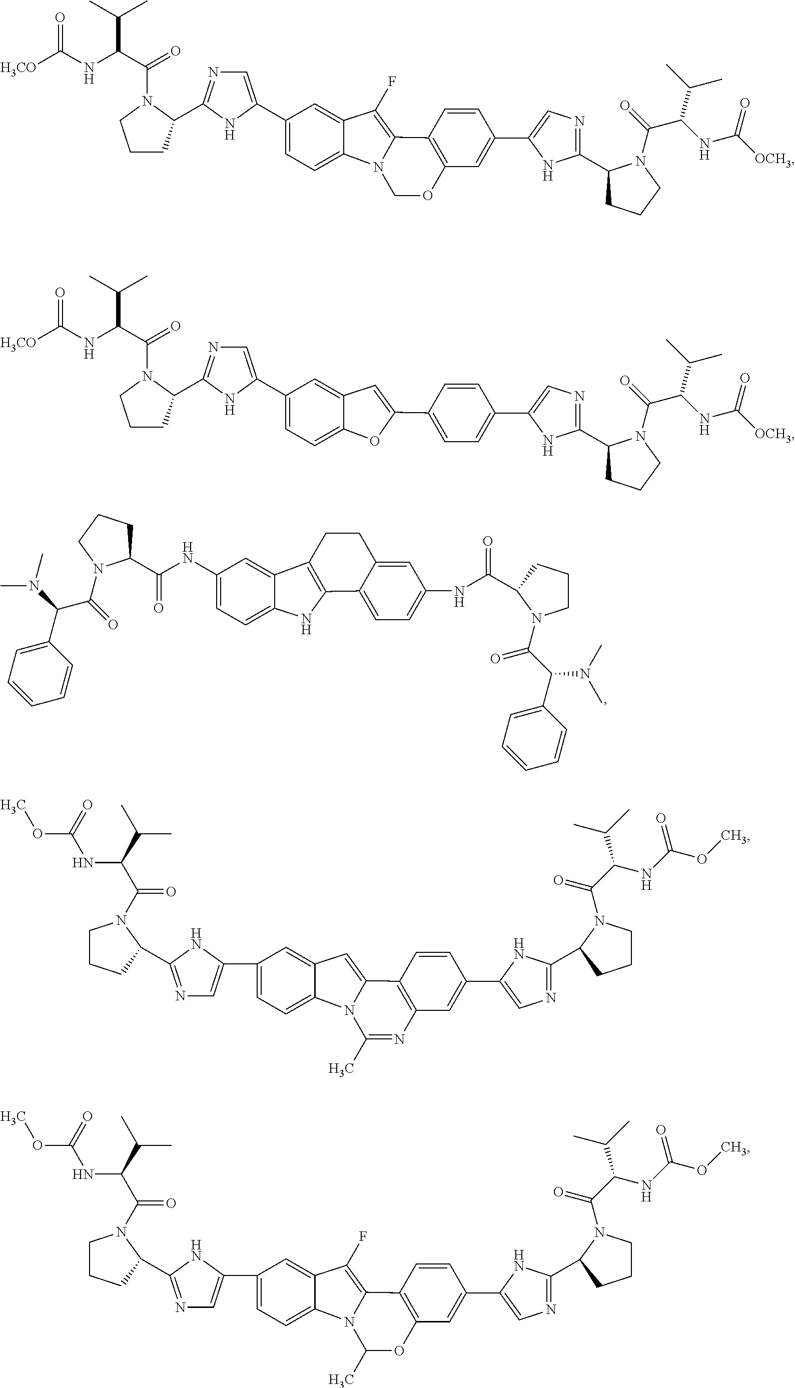 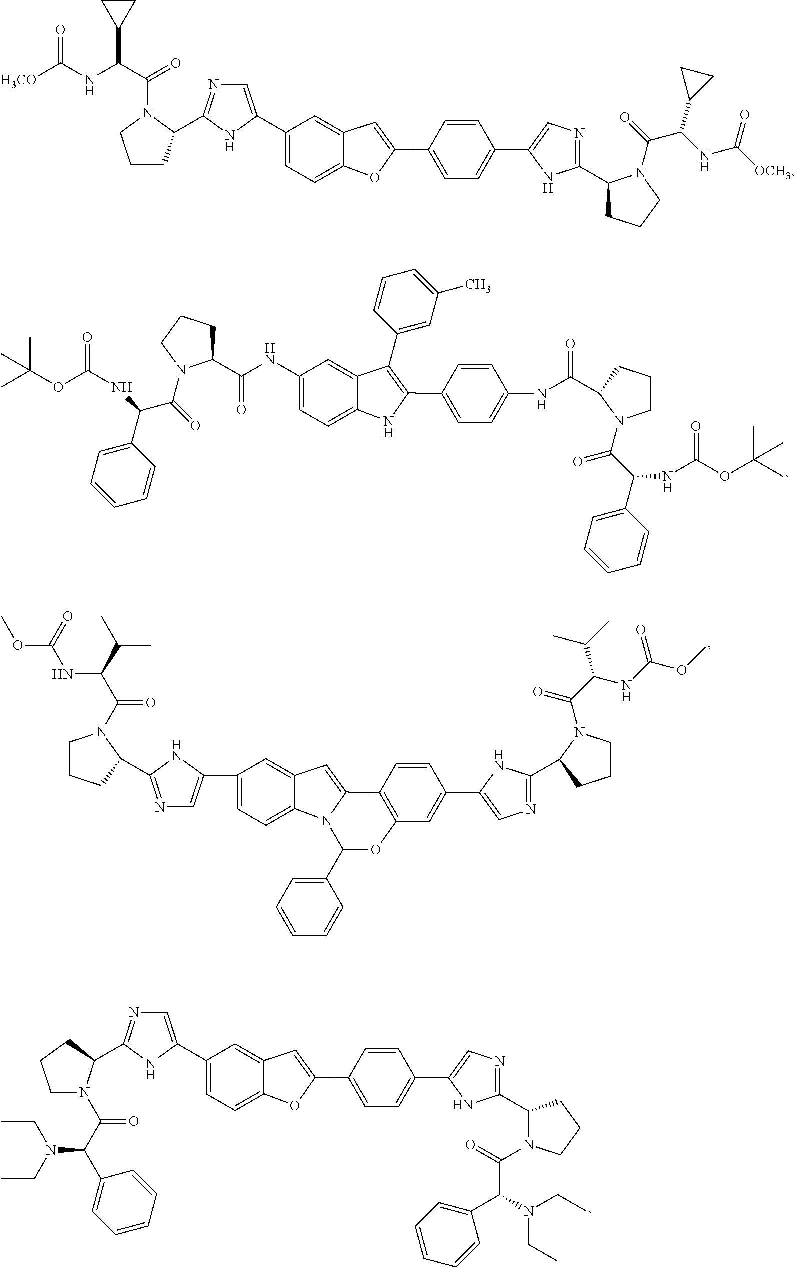 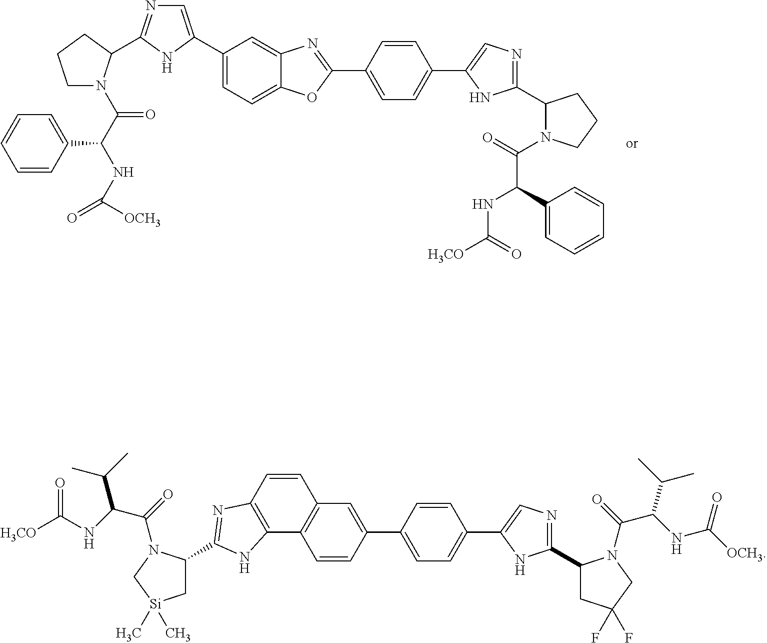
or a pharmaceutically acceptable salt thereof.

2. The pharmaceutical composition of claim 1, wherein the one or more Primary Additional Therapeutic Agents are selected from an HCV polymerase inhibitor and an HCV NS5A inhibitor.

3. The pharmaceutical composition of claim 1, wherein the one or more Primary Additional Therapeutic Agents comprise an HCV polymerase inhibitor.

4. The pharmaceutical composition of claim 1, wherein the one or more Primary Additional Therapeutic Agents comprise an HCV NS5A inhibitor.

5. The pharmaceutical composition of claim 1, further comprising one or more Secondary Additional Therapeutic Agents.

6. The pharmaceutical composition of claim 1, wherein the one or more Primary Additional Therapeutic Agents comprise PSI-7977, PSI-938 or R7128.

7. The pharmaceutical composition of claim 1, wherein the one or more Primary Additional Therapeutic Agents comprise BMS-790052.

8. The pharmaceutical composition of claim 1, wherein the Secondary Additional Therapeutic Agent comprises one or both of ribavirin and pegylated interferon-α.

9. A method of treating a patient infected with HCV, the method comprising administering to the patient: (i) a compound selected from Table 1:
TABLE 1

| | |
|---|---|
| 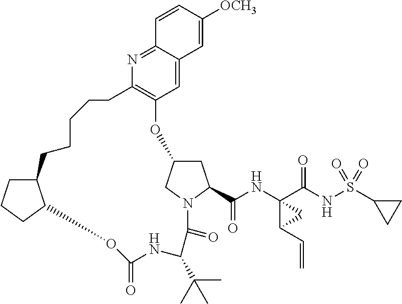 | 1 |
| 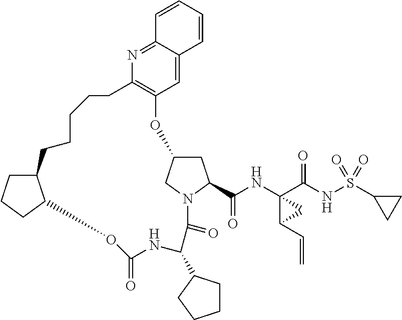 | 2 |
| 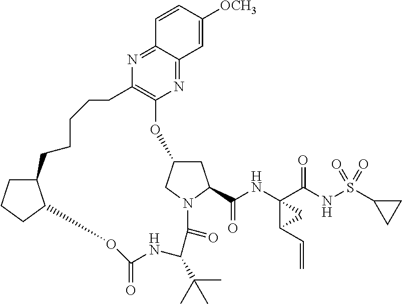 | 3 |
| 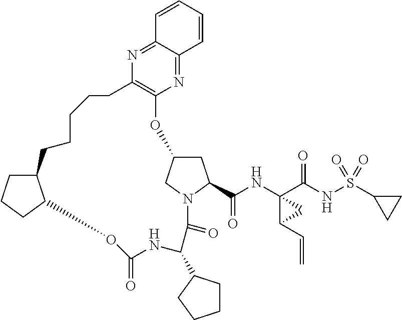 | 4 |
| 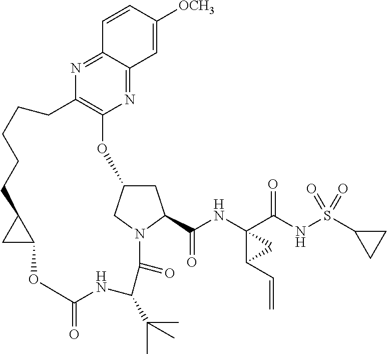 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)NS(=O)(=O)C1CC1] | 5 |
| 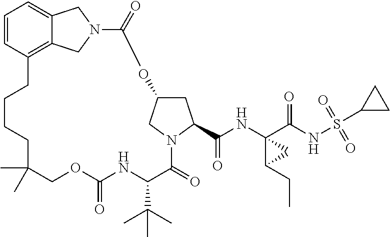 [SMILES: CC[C@@H]1C[C@]1(NC(=O)[C@@H]1C[C@@H]2CN1C(=O)[C@H](C(C)(C)C)NC(=O)OCC(C)(C)CCCCc1cccc3c1CN(C3)C(=O)O2)C(=O)NS(=O)(=O)C1CC1] | 6 |
| 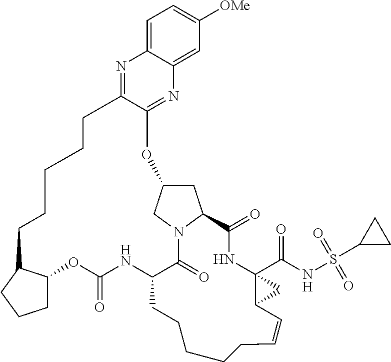 | 7 |
| 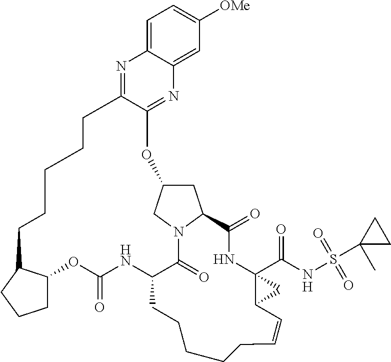 | 8 |
| 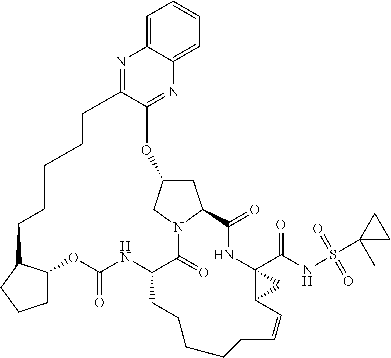 | 9 |
| 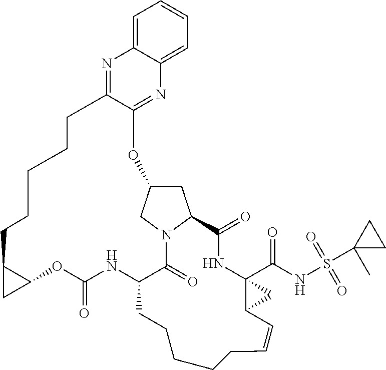 [SMILES: CC1(S(=O)(=O)NC(=O)[C@@]23C[C@H]2/C=C\CCCCC[C@@H]2NC(=O)O[C@@H]4C[C@H]4CCCCCc4nc5ccccc5nc4O[C@@H]4C[C@@H](C(=O)N3)N(C4)C2=O)CC1] | 10 |
| 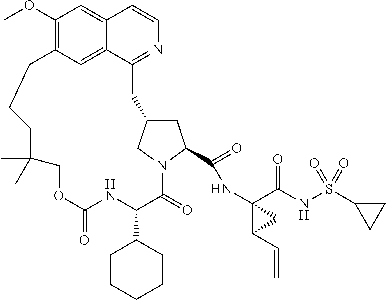 [SMILES: C=C[C@@H]1C[C@]1(NC(=O)[C@@H]1C[C@H]2Cc3nccc4cc(OC)c(cc34)CCCC(C)(C)COC(=O)N[C@@H](C3CCCCC3)C(=O)N1C2)C(=O)NS(=O)(=O)C1CC1] | 11 |
| 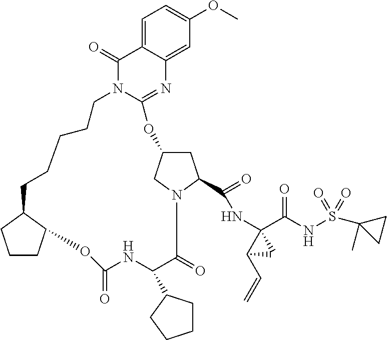 [SMILES: C=C[C@@H]1C[C@]1(NC(=O)[C@@H]1C[C@@H]2CN1C(=O)[C@H](C1CCCC1)NC(=O)O[C@@H]1CCC[C@H]1CCCCCn1c(nc3cc(OC)ccc3c1=O)O2)C(=O)NS(=O)(=O)C1(C)CC1] | 12 |
| 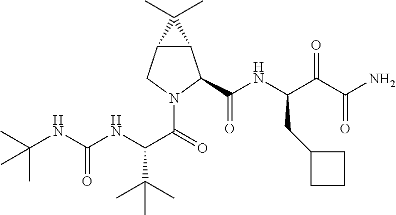 [SMILES: CC(C)(C)NC(=O)N[C@H](C(=O)N1C[C@H]2[C@@H]([C@H]1C(=O)N[C@H](CC1CCC1)C(=O)C(N)=O)C2(C)C)C(C)(C)C] | 13 |
| 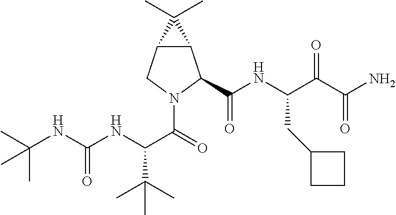 [SMILES: CC(C)(C)NC(=O)N[C@H](C(=O)N1C[C@H]2[C@@H]([C@H]1C(=O)N[C@@H](CC1CCC1)C(=O)C(N)=O)C2(C)C)C(C)(C)C] | 14 |

or a pharmaceutically acceptable salt thereof; and (ii) one or more additional therapeutic agents, or a pharmaceutically acceptable salt thereof, wherein the one or more additional therapeutic agents are selected from the group consisting of HCV protease inhibitors, HCV polymerase inhibitors, HCV NS4A inhibitors and HCV NS5A inhibitors, wherein the one or more additional therapeutic agents do not comprise a Compound of Table 1, and wherein the amounts administered of the Compound of Table 1 and the one or more additional therapeutic agents are together effective to treat the HCV infection in said patient, such that when the one or more additional therapeutic agents comprise an HCV NS5A inhibitor, the HCV NS5A inhibitor is not one of the following compounds: 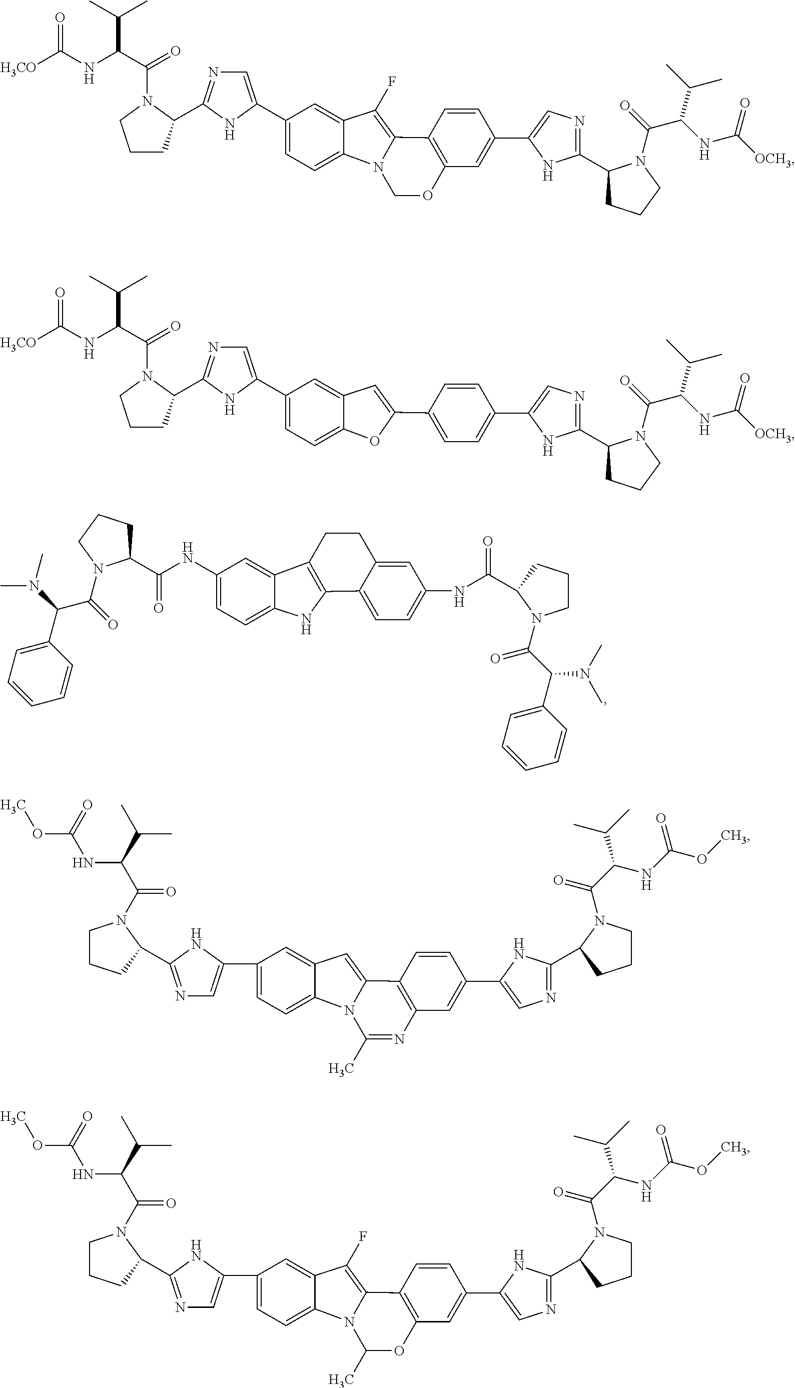 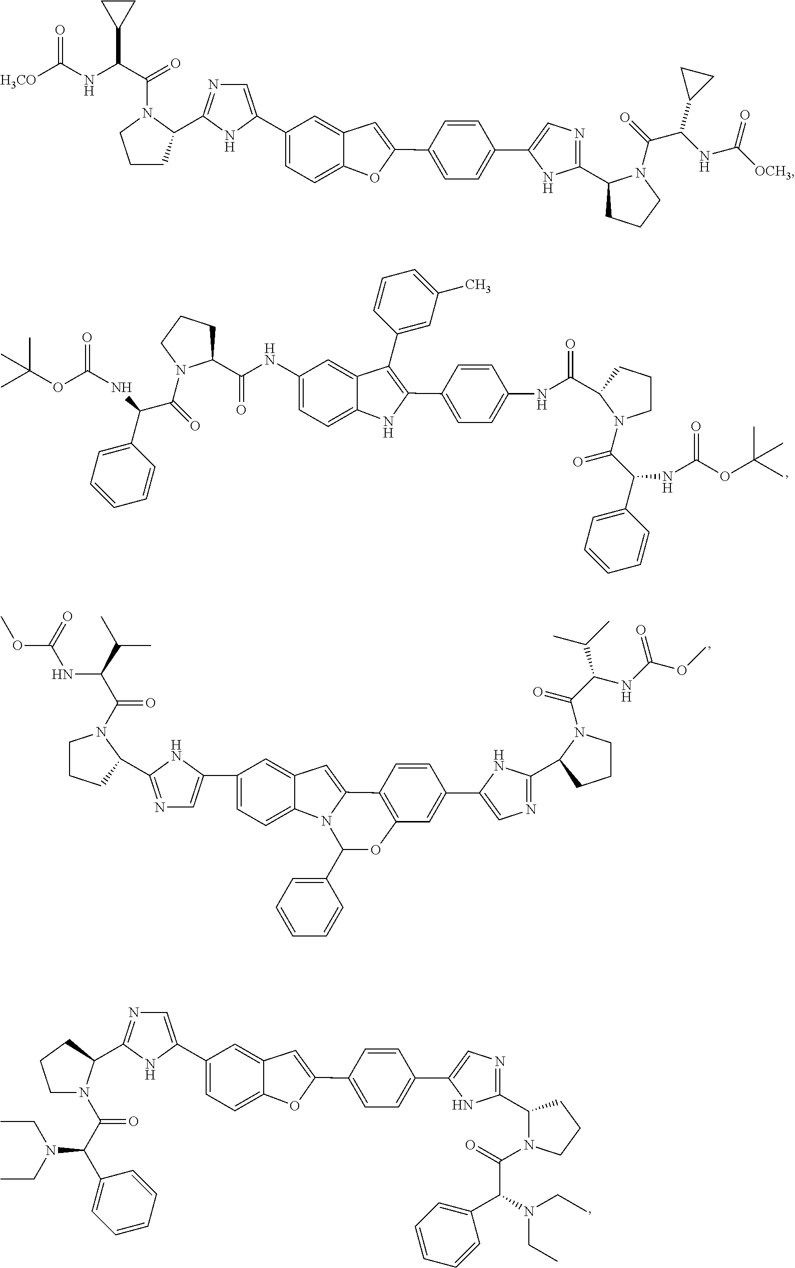 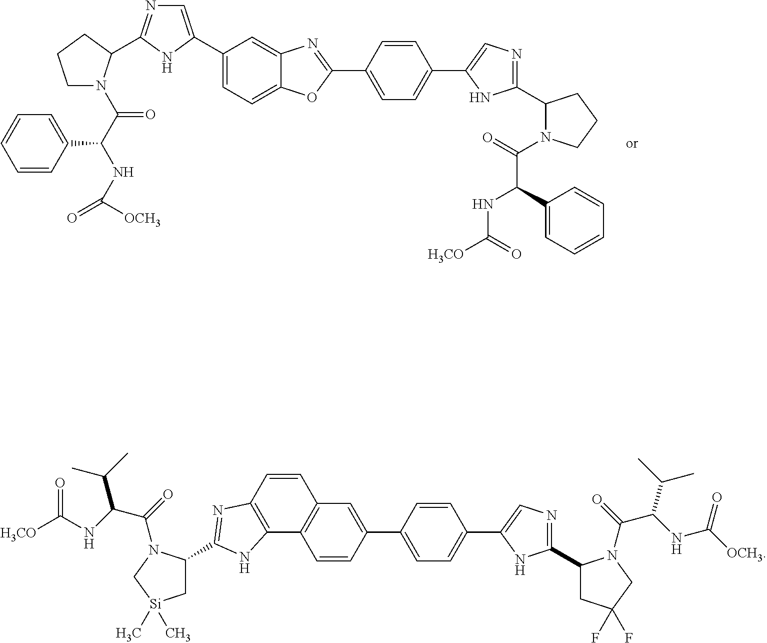
or a pharmaceutically acceptable salt thereof.

10. A method of treating a patient infected with HCV, the method comprising administering to the patient the composition of claim 1.

11. A method for inhibiting HCV replication or for preventing and/or treating infection by HCV in a patient in need thereof, comprising providing a composition of claim 1.


SEQUENCE LISTING (3 sequences)

SEQ ID NO: 1 - DNA, 15 bp, Artificial Sequence
  5B.2F Primer
ccggctactgcccat

SEQ ID NO: 2 - DNA, 22 bp, Artificial Sequence
  5B.2F Primer
ccagatcatcctgatcgacaag

SEQ ID NO: 3 - DNA, 24 bp, Artificial Sequence
  FAM Labeled Probe
acatcgcatcgagcgagcacgtac